(19) United States
(12) Patent Application Publication
(10) Pub. No.: US 2024/0010983 A1
(43) Pub. Date: Jan. 11, 2024

(54) CONSUMABLE TISSUE-LIKE STRUCTURE GENERATED WITH MUSCLE CELLS GROWN ON EDIBLE HOLLOW FIBERS

(71) Applicant: Merck Patent GmbH, Darmstadt (DE)

(72) Inventors: Jean-Louis Weissenbach, Strasbourg, MA (US); Ryan Sylvia, Tewksbury, MA (US); Almut Von der Brelie, Frankfurt am Main (DE); Melanie Brandl, Ober-ramstadt (DE); Michaela Fesenfeld, Darmstadt (DE)

(21) Appl. No.: 18/042,037

(22) PCT Filed: Aug. 19, 2021
(86) PCT No.: PCT/EP2021/073078

§ 371 Date: Feb. 17, 2023

Related U.S. Application Data
(60) Provisional application No. 63/068,397, filed on Aug. 21, 2020.

(51) Int. Cl. C12N 5/077; A23J 3/18; A23J 3/04; A23J 3/22; C12M 1/12; C12M 1/36

(52) U.S. Cl. CPC C12N 5/0658; A23J 3/18; A23J 3/04; A23J 3/227; C12M 25/10; C12M 41/48; C12N 2533/74; C12N 2533/50

(57) ABSTRACT

The present invention is directed toward edible hollow fibers and cartridges and bioreactors comprising the hollow fibers of the present invention, as well as, methods of production of structured clean meat products produced with the hollow fibers, cartridges and bioreactors of the present invention and the structured clean meat products produced by said methods. The macroscopic structure of structured clean meat grown on edible hollow fibers will result in a unique final structure. This final structure will contain a finite amount of fibers per unit area; with meat on the outside of the fibers.

### BACKGROUND OF THE INVENTION

Lab grown meat or meat-like products, often referred to as “clean meat,” are likely to be a significant player in providing food for the ever-increasing human population. However, efforts in this field have only met with limited success. Part of the reason is because the product generated with current technology has little to no structure, resembling, at best, a ground meat-like substance that must be combined with other ingredients to give it meat-like structure and texture.

In order to generate structured clean meat, scaffolding must be used. To be practicable and cost effective, the scaffolding must be edible and/or dissolvable and result in a texture and structure in the final product that gives a mouth feel reminiscent of real meat or natural meat (i.e., meat derived from an animal). This means that the scaffolding must provide for at least three qualities: 1) be edible, 2) provide a texture and mouth feel similar to real meat and, 3) be a suitable culture environment for myocytes or myocyte-like cells and other cell types to grow efficiently and form muscular structure (for example, form myotubules and achieve tissue-like cell densities) resembling natural muscle.

Obtaining each of these goals has been a challenge in the art. Obtaining all three of these goals in one culture system has not been achieved in the art in a large part because achieving any one of the three goals can work against achieving one or both of the others.

The reasons for this are many-fold. We will review a few of them here. Edible microcarriers have been tried but edible microcarriers cannot achieve tissue like cell densities or provide a meat-tissue mouth feel or texture without an additional processing step and/or material (see, e.g., US Patent Publication No. 2015/0079238 to Marga).

Hydrogel tubes have been used. One benefit is that myocytes are able to form myotubules on hydrogel tubes. However, cell densities are not high enough and the final macroscopic structure does not resemble natural meat. Further processing would be required, e.g., for myotube alignment (see, Qiang, Li, et al., 2018 Biofabrication 10:025006).

Three-dimensional (3D) hydrogel matrices are well known and commercially available. Unfortunately, this format is not well suited for tissue-like cell densities, This is because, at least in part, vascularization/media diffusion is limited (Bramfeldt, et al., Curr Med Chem. 2010; 17(33):3944-3967). Additionally, the structure would give an unnatural sponge-like mouth feel.

Three-dimensional printing has been undertaken by companies such as Redefine Meat, Ltd. (Ness Ziona, Israel) and progress has been made in the structure and cell density aspects of assembling a nonmeat-based structured meat-like product. However, this approach is a finishing step. The majority of the cell growth would need to be completed ahead of time and the cells processed into the 3D structure adding excessive time and cost to the process.

Decellularized plant materials (e.g., celery and rhubarb) have been described as a scaffolding material (see, Gershlak, J., et al., Biomaterials, 2017 May; 125:13-22). However, fluid management becomes a challenge beyond lab scale making any process using decellularized pant material difficult to scale-up to production scale. Further, the final structure mirrors the plant material making the mouth feel and texture unsatisfactory for a meat-like product. While decellularized plant material seems to allow for the correct orientation of cells and aids in vascularization of the cultured cells, scale-up has proven to be a critical hurdle to be overcome. Further, mouth feel may more closely resemble vegetative matter than natural meat. See, e.g., Decellularized Plant-based Scaffold for Guided Alignment of Myoblast Cells—Santiago Campuzano, et al., 2020, in press and www.new-harvest.ordsantiago_campuzano.

Plant based hollow fibers may be problematic with regard to providing an edible product. Whole grain starches are edible but dissolve in cell culture media and, therefore, are not suitable for cell culture or cell adherence. Cellulose has been widely used in the filtration industry. Cellulose is recognized as a material within existing food, but in a membrane format cellulose is typically chemically modified and behaves like a plastic. This macroscopic behavior of cellulose makes it undesirable for consumption. Likewise, collagen blended hollow fibers have been described. However, these products were not designed for human consumption or for use in the clean meat industry (see, WO2018162857 A1, Hollow Cellular Microfibre and Method for Producing Such a Hollow Cellular Microfibre) and are likely unsuitable for use in this area.

Further still, existing edible scaffolding does not allow for vascularization which is required for generating structured meat thicker than several hundred microns. An example of edible scaffolding, as noted above, are microcarriers. Microcarriers must be either suspended in a culture vessel or packed bed or bulk material form. In these cases, the cell culture is fed only from the medium that the scaffold is submerged in. Therefore, the culture growth is limited by the diffusion of media in the tissue, which is reported to be less than 200 μm.

Scaffolding has also been reported in essentially two-dimensional formats such as electrospun sheets of material (see, e.g., Yang, F., et al., Materials (Basel), 2017 Oct. 12; 10 (10)). These two-dimensional sheets can be fed by diffusion of the media through the sheet as well as by the surrounding media.

In addition, the orientation of the muscle tissue grown on prior art scaffolds does not orient correctly to include the formation of muscle fibrils. Scaffolding as needed and described for producing an edible, structured clean meat product is not known in the art nor commercially available.

Thus, there is a current need for improved cell culture materials, as well as devises and methods employing those materials, for the generation of structured clean meat products.

### SUMMARY OF THE INVENTION

The present invention is directed toward cell culture devices and systems that provide an edible clean meat product comprising a scaffold that is 1) edible, 2) provides a texture, structure and mouth feel similar to real meat and, 3) promotes the growth of myocytes and other cell types into structures resembling natural muscle. Further, the scaffold and clean meat growth thereon has texture and handling qualities that resemble natural meat during, for example, processing (i.e., cutting or slicing) and cooking. In other words, the present invention provides for materials and methods suitable for producing a textured, structured meat product. The present invention achieves these goals through a culture system based on edible hollow fibers, culture devices utilizing the edible hollow fibers of the present invention and culture methods utilizing the culture devices of the present invention. The hollow fibers of the present invention may also be partly or completely dissolvable, as discussed infra.

The present inventors have found that by using the edible hollow fibers of the present invention, cost effective structured clean meat can be generated. The hollow fibers of the present invention add to the quality of the end product of the structured clean meat in terms of muscle structure as well as other desirable properties. Further, by filling the lumen of the hollow fibers with a lipid-based component at the end of the culture period, the fat content of the desired end product can be tuned and homogeneously distributed throughout the product.

The hollow fiber composition of the present invention can also aid in the mouth feel of the end product as well as the taste of the end product. Further, flavors can be added to the center of the hollow fibers at the end of the culture period with, for example, a colloidal material or other material. The colloidal material may be liquid or slurry comprising one or both of lipids (fats) and flavors, as well as other ingredients.

By using a variability of base materials to create the hollow fiber, the flavor and texture of the structured clean meat end product can be influenced. In this sense, the hollow fibers of the present invention can be customized depending on the desired end product. For example, the materials used to make the hollow fibers can be adjusted depending on the type of meat being grown and spacing (i.e., between hollow fibers and diameter of the hollow fibers) can be modified to give, for example, different final textures. One of skill in the art, with the teachings of this specification, would be able to modify the hollow fibers and any associated hollow fiber apparatus (e.g., a hollow fiber cartridge) of the present invention to influence characteristics of the final end product, as desired.

Cell growth characteristics can also be controlled based on the structure and composition of the hollow fibers of the present invention. For example, utilizing fibrous components in the construction of the hollow fibers, rather than particles or particulates, is likely to result in different cell adherence characteristics and, ultimately, different final product textures both of the fibers and the final clean meat product. For example, utilizing fibrous components in the construction of the hollow fibers of the present invention is understood by the inventors to orient cell growth along the fibers. Similarly, fibers with a textured surface may influence the final product. Further still, the fibers of the present invention can have factors (e.g., growth factors, attachment factors) incorporated into or applied onto the surface of the hollow fibers of the present invention. Some of the above discussed modifications to the hollow fibers of the present invention also can influence one or more of the taste, the structure, the strength, the texture and the mouth feel of the final product.

Herein is provided the invention of novel and non-obvious hollow fibers suitable for the uniform or substantially uniform growth of cellular materials (i.e., cells such as, but not limited to, muscle cells, fat cells and fibroblasts) to a uniform or substantially uniform density along an individual hollow fiber and along hollow fibers arranged is a desired pattern, as well as with a uniform or substantially uniform distribution of void space between said arranged hollow fibers. The present invention is also related to bioreactors utilizing, at least in part, the hollow fibers of the present invention as well as processes and methods of making structured clean meat products and the structured clean meat products made with the hollow fibers of the present invention. While the present invention is not limited to theory, it is believed that the surprising and unexpected characteristics of the hollow fibers of the present invention can be attributed at least to the combination of fiber material(s), fiber configuration, fiber porosity and fiber dimensions. Further, these characteristics of the hollow fibers of the present invention are believed to work in synergy with and in combination with the cell type or cell types being cultured in the production of the structured clean meat product of the present invention.

In one aspect, the present invention is directed toward edible hollow fibers comprising one or more materials selected from the group consisting of hydrocolloids and proteins, having an outer diameter of about 0.2 mm to about 2.0 mm, a porosity of 0% to about 75% and a wall thickness of about 0.05 mm to about 0.4 mm. In another aspect, the hollow fibers have a wall thickness is about 0.08 mm to 0.2 mm. In another aspect, the hollow fibers have a porosity is about 40% to about 60%.

In another aspect, the hollow fibers of the present invention comprise one or more of alginate, collagen, cellulose, chitosan, collagen, zein, agar, inulin, gluten, pectin, legume protein, methyl cellulose, pectin, gelatin, tapioca, xanthan gum, guar gum, tara gum, bean gum, plant protein, starch, plant isolates (e.g., soy/zein/casein/wheat protein), lipids, (e.g., free fatty acids, triglycerides, natural waxes, and phospholipids. In another aspect of the present invention the proteins of the hollow fibers comprise one of more of corn protein, potato protein, wheat protein, sorghum protein, animal protein, animal protein isolate, beef protein isolate, casein protein and whey protein. In another aspect of the present invention, the plant isolates of the hollow fibers comprise one of more of soy, zein, casein, and wheat protein. In another aspect of the present invention, the lipids of the hollow fibers of the present invention comprise one or more of free fatty acids, triglycerides, natural waxes and phospholipids. In yet another aspect of the present invention, the hollow fibers comprise one or more legume proteins and one or more hydrocolloids.

In another aspect of the present invention, each hollow fiber of the present invention has a first end and a second end wherein the first end and the second end are positionally opposed to each other and, wherein a quantity of the hollow fibers are arranged in parallel or essentially in parallel and positioned such that the first ends of the hollow fibers are secured in a first holding device and the second ends of the hollow fibers are secured in a second holding device, the first and second holding devices being oriented perpendicular or essentially perpendicular to the longitudinal orientation of the hollow fibers and being orientated parallel or essentially parallel to each other, wherein at least one holding device allows for the flow of fluids to the interior of the hollow fibers, thereby creating a hollow fiber cartridge. In another aspect of the present invention, the hollow fibers of the hollow fiber cartridge are at a density of about 40-about 120 per cm². In another aspect of the present invention, the hollow fibers of the hollow fiber cartridge are at a density of about 60-about 100 per cm². In yet another aspect of the present invention, the hollow fibers of the hollow fiber cartridge are at a density of about 70-about 90 per cm².

In another aspect of the present invention, the hollow fiber cartridge of the present invention has a void space between the hollow fibers and the void space between the hollow fibers is about 25%-about 75% of the total volume of the hollow fiber cartridge. In yet another aspect of the present invention, the void space between the hollow fibers is about 40%-about 60% of the total volume of the hollow fiber cartridge.

In another aspect of the present invention, the hollow fiber cartridge is designed to be removably inserted into a housing. In another aspect of the present invention, the housing is part of a bioreactor or bioreactor system.

In another aspect, the present invention is directed toward a hollow fiber cell culture reactor comprising a hollow fiber cell culture cartridge of the present invention, a housing sized to hold the hollow fiber cartridge of the present invention, a cell culture medium source fluidly connected to one or more inlets in the housing, one or more outlets in the housing and, one or more pumps to supply the medium to and/or remove waste medium and/or gases from the hollow fiber cartridge through the medium inlet(s) and/or outlet(s). In another aspect of the present invention, the inlets are fluidly connected to the interior of the hollow fibers. In another aspect of the present invention the inlets are fluidly connected to the void space between the hollow fibers and the outlets are fluidly connected to the interior of the hollow fibers, thereby creating a fluid flow from the outside to the inside of the hollow fibers. In yet another aspect of the present invention, the hollow fiber cell culture reactor comprises an automated controller. The automated controller may include a computer.

In an aspect of the present invention, the present invention is directed toward a process for producing a meat product, comprising; seeding the void space between the hollow fibers in the hollow fiber reactor of the present invention with one or more of myocytes, myocyte-like cells or engineered cells expressing one or more myocyte-like characteristics at a density of about 10⁵ cells/ml to about 10⁸ cells/ml and culturing the cells until achieving about 80%-about 99% confluency; about 85%-about 99% confluency; or about 90%-about 99% confluency. In another aspect of the present invention, the process additionally comprises removing the hollow fiber cartridge from the hollow fiber cell culture reactor after the cells have achieved desired confluency. In another aspect of the present invention, the process of the present invention additionally comprises removing the first holding device and the second holding device from the first ends and second ends, respectively, of the hollow fibers. In another aspect of the present invention, the process of the present invention additionally comprises seeding (i.e., in addition to the myocytes, myocyte-like cells or engineered cells expressing one or more myocyte-like characteristics discussed, supra) the hollow fiber reactor with one or more of adipocytes, adipocyte-like cells or engineered cells expressing one or more adipocyte-like characteristics and/or fibroblasts, fibroblast-like cells or engineered cells expressing one or more fibroblast-like characteristics.

In another aspect of the present invention, the process of the present invention additionally comprises supplying media to the cells through one or both of the first end and second end of the hollow fibers into the interior of the hollow fibers, through the wall of the hollow fibers into the void space between the hollow fibers where the cells are seeded and through one or more of said outlets in said housing. In another aspect of the present invention, the flow of media is reversed for at least a portion of the culture process.

In another aspect of the present invention, the process of the present invention further comprises that after the cells achieve confluency, the interior of the hollow fibers and/or any remaining void space between the cells is infused with one or more of fats, flavors, colors, salts and preservatives.

In another aspect of the present invention, the present invention relates to a structured clean meat product, comprising: a) 50-90% cultured animal cells; b) 10-30% edible hollow fibers and/or hollow fiber material; c) 1-30% void space, the void space located between and/or interspersed with the cultured animal cells; and d) 1-30% additives. The structured clean meat product may be produced by the process of the present invention using the hollow fiber cell culture reactor and hollow fibers of the present invention.

In an aspect of the present invention, the additives added to the structured clean meat product of the present invention comprise one or more of flavors, texture enhancers, nutritional additives, preservatives, and fats. In an aspect of the invention, the flavors are selected from one or more of essential oils, oleoresin (HO), enzymes (ENZ), natural substances and extractives (NAT), non-nutritive sweetener (NNS), nutritive sweetener (NUTRS), spices, natural seasonings & flavorings (SP), and synthetic flavors (SY/FL), fumigant (FUM), artificial sweeteners and yeast extract. In another aspect of the present invention, the texture enhancers are selected from one or more of pureed plant material, guar gum, cellulose, hemicellulose, lignin, beta glucans, soy, wheat, maize and rice isolates and beet fiber, pea fiber, bamboo fiber, plant derived fiber, plant derived gluten, carrageenan, xanthan gum, lecithin, pectin, agar, alginate, natural polysaccharides, grain husk, calcium citrate, calcium phosphates, calcium sulfate, magnesium sulfate and salts. In another aspect of the present invention, the nutritional additives are selected from one or more of trace elements, bioactive compounds, endogenous antioxidants, A, B-complex, C, D, E vitamins, zinc, thiamin, riboflavin, selenium, iron, niacin, potassium, phosphorus, omega-3, omega-6, fatty acids, magnesium, protein, amino acids salt, creatine, taurine, carnitine, carnosine, ubiquinone, glutathione, choline, glutathione, lipoic acid, spermine, anserine, linoleic acid, pantothenic acid, cholesterol, Retinol, folic acid, dietary fiber and amino acids. In yet another aspect of the present invention, the fats are selected from one or more of saturated, monounsaturated, polyunsaturated fats, corn oil, canola oil, sunflower oil, safflower oil, olive oil, peanut oil, soybean, flax seed oil, sesame oil, canola oil, avocado oil, seed oils, nut oils, safflower and sunflower oils, palm oil, coconut oil, omega-3, fish oil, lard, butter, processed animal fat, adipose tissue, cellular agriculture derived fat essential oil and oleoresin. In another aspect of the present invention, the preservative and/or antioxidant is selected from one or more of: sodium salt, chloride salt, iodine salt. Nitrates, nitrosamines, butylated hydroxyanisole (BHA), butylated hydroxytoluene (BHT), sodium benzoate, potassium benzoate and benzene ascorbic acid, citric acid, potassium, monosodium glutamate (MSG), sulphur dioxide, sulphites, antibiotics. It is noted here that any one additive, flavor, texture enhancer, nutrient additive, fat/oil and/or preservative/antioxidant may supply more than one attribute to the structured clean meat product of the present invention.

In an aspect of the present invention, the hollow fibers of the structured clean meat product of the present invention the hollow fibers comprise one or more legume proteins and one or more hydrocolloids. In another aspect of the present invention, the void space is void of cells and/or cellular material. In yet another aspect of the present invention, the void space is at least partly filled with material other than cells or cellular material.

### BRIEF DESCRIPTION OF THE FIGURES

FIGS. 1(A & B) show certain embodiments of the geometric arrangement of the hollow fibers of the present invention. FIG. 1A shows a rectangular pattern and FIG. 1B shows a triangular pattern. Figures are for illustrative purposes of the fiber arrangement only. Fiber spacing in an actual hollow fiber cartridge of the present invention could have greater void space between the fibers than illustrated. Likewise, the spacing between individual hollow fibers may vary.

FIGS. 2(A & B) show certain embodiments of an end view representation of cell growth on the hollow fibers of the present invention. Figure shows that in some instances, even after cell growth, a minimum of void space may still be present.

FIG. 3 shows a representation of an embodiment of a cross section of a single hollow fiber of the present invention with A being the center or lumen of the hollow fiber, B being the porous hollow fiber wall and C being the cell mass.

FIG. 4 shows exemplary calculated percentages of clean meat (“meat”), fiber and void space based on different fiber diameters (OD=outer diameter). One of skill in the art will be able to extrapolate from these figures for fibers of larger or narrower ODs.

FIG. 5 shows exemplary calculated percentages of clean meat (“meat”), fiber and void space for three different fiber ODs.

FIG. 6 presents the exemplary data from FIG. 5 in table format.

FIG. 7 shows exemplary calculated data on embodiments of the hollow fibers of the present invention in table format.

FIG. 8 shows dissolved alginate:protein mixtures. From left to right: soy acid hydrolysate; beef protein isolate, whey protein isolate, brown rice protein isolate, pea protein isolate and soy protein isolate.

FIG. 9 shows various alginate alone (without protein). Large jars have been autoclaved; small jars have not been autoclaved.

FIGS. 10(A-D) shows hollow fibers made as per the Exemplification. (A) shows fibers after production. (B) & (C) show fibers at 100× magnification. (D) shows another fiber at 50× magnification.

FIG. 11 shows that the hollow fibers of the present invention can easily support their weight, which will be needed in a bioreactor. The fiber shown is 2 meters long.

FIGS. 12(A & B) shows cryogenic scanning electron micrographs (SEM) of a hollow fiber of the present invention made from a mixture of 5:2 whey protein:alginate. (A) 100×. (B) 5000×.

FIGS. 13(A-D) shows increasing magnifications of the hollow fibers made from 5:2 whey protein:alginate. (A) scale bar=10 μm, (B) scale bar=1 μm, (C) scale bar=1 μm (larger scale bar as compared to FIG. 13B indicates that the magnification is approximately 3× that of FIG. 13B), (D) scale bar=100 nm. These samples were made using critical point drying followed by SEM.

FIGS. 14(A & B) shows cryogenic SEM of a hollow fiber of the present invention made from a mixture of soy protein:alginate. (A) 100×. (B) 5000×.

FIGS. 15(A & B) shows increasing magnification of the hollow fibers made from 5:2 soy protein:alginate. (A) scale bar=10 μm, (B) scale bar=1 μm. These samples were made using critical point drying followed by SEM.

FIG. 16 shows (A & B) shows further increasing magnification of the hollow fibers made from 5:2 soy protein:alginate. (A) shows a different area than FIG. 15A: scale bar=1 μm, (B) scale bar=200 nm. These samples were made using critical point drying followed by SEM. Also of note is the bacterial contamination seen in FIG. 16A, indicative of the perishable nature of the edible hollow fibers of the present invention.

FIG. 17 shows (A & B) shows cryogenic SEM of a hollow fiber of the present invention made from a mixture of pumpkin protein:alginate. (A) 1000×. (B) 10,000×.

FIG. 18 shows (A & B) shows cryogenic SEM of a hollow fiber of the present invention made from a mixture of pumpkin protein:alginate. (A) 20,000×. (B) 40,000×.

FIG. 19 shows (A-D) shows increasing magnifications of the hollow fibers made from 5:2 pumpkin protein:alginate. (A) scale bar=10 μm, (B) scale bar=1 μm, (C) scale bar=100 nm, (D) scale bar=1 μm. These samples were made using critical point drying followed by SEM.

FIG. 20 shows (A & B) shows cryogenic SEM of a hollow fiber of the present invention made from a mixture of pea protein:alginate. (A) 100×. (B) 5000×.

FIG. 21 shows (A & B) shows cryogenic SEM of a hollow fiber of the present invention made from a mixture of beef protein:alginate. (A) 1000×. (B) 10,000×.

FIG. 22 shows (A & B) shows cryogenic SEM of a hollow fiber of the present invention made from a mixture of beef protein:alginate. (A) 20,000×. (B) 40,000×.

FIG. 23 shows (A & B) shows further increasing magnification of the hollow fibers made from 5:2 beef protein:alginate. (A) scale bar=1 μm, (B) scale bar=1 μm. These samples were made using critical point drying followed by SEM.

FIG. 24 shows (A) scale bar=1 urn, (B) scale bar=100 nm. These samples were made using critical point drying followed by SEM. 1 μm scales from FIGS. 23A, 23B and 24A are from different areas of the hollow fiber.

FIG. 25 shows (A & B) shows cryogenic SEM of a hollow fiber of the present invention made from a mixture of rice protein:alginate. (A) 1000×. (B) 5000×.

FIG. 26 shows cell growth of C2C12 murine muscle cells on hollow fibers of the present invention. Cell growth is relative as measured by raw luminescent units (RLU) indicating ATP generation. See, Example 1C.

FIGS. 27(A-C) show cells growing and attached to hollow fibers of the present invention and as described in Example 1C. (A) shows staining for both live and dead cells. (B) shows staining of live cells. (C) shows staining of dead cells.

### DETAILED DESCRIPTION OF THE INVENTION

The present invention contemplates edible and/or dissolvable hollow fibers, bioreactors comprising the hollow fibers of the present invention for the production, for example, of structured clean meat, and methods of production of structured clean meat therewith and the structured clean meat produced with the hollow fibers of the present invention. Clean meat is defined in the art as meat or a meat-like product (referred to collectively herein as “clean meat” or “clean meat product”) grown from cells in a laboratory, factory or other production facility suitable for the large-scale culture of cells.

A “structured meat product” or “structured clean meat product” is a meat product or clean meat product having a texture and structure like, similar to or suggestive of natural meat from animals. The structured meat product of the present invention has a texture and structure that resembles natural meat 1) in texture and appearance, 2) in handleability when being prepared for cooking and consumption (e.g., when being sliced, ground, cooked, etc.) and 3) in mouth feel when consumed by a person. The materials and methods of the present invention, when used in the production of structured clean meat, achieve at least one of these criteria, two of these criteria or all three of these criteria. The prior art technology is unable to produce a structured meat product sufficiently meeting any of these criteria.

The structured meat product of the present invention meets these criteria by culturing suitable cells (discussed, infra) in a bioreactor (also, discussed, infra) comprising the hollow fibers of the present invention. The hollow fibers of the present invention, at least in considerable part, provide the structure and texture to the final structured clean meat product that provides the desired appearance, handleability and mouth feel of the product. Further, the hollow fibers of the present invention aid in providing a suitable environment for the growth of the cells into a structured clean meat product. In this context, the hollow fibers of the present invention provide at least a surface suitable for the attachment of the cultured cells, elongation of the cells into morphologies resembling myocytes or myocyte-like cells (i.e., substantially resembling myocytes in structure and appearance), and formation of the myocytes into myotubule or myotubule-like structures (i.e., substantially resembling myotubules in structure and appearance).

It is contemplated that the edible and/or dissolvable hollow fibers of the present invention are made from one or more of hydrocolloids (such as Xanthan, methyl cellulose(s), alginate, agar, pectin, gelatin, Guar/Tara/Bean/other gums), proteins (e.g., polypeptides, peptides, glycoprotein and amino acids; for example, various starches (corn/potato/rice/wheat/sorghum), plant isolates (e.g., soy/zein/casein/wheat protein), lipids, (e.g., free fatty acids, triglycerides, natural waxes, and phospholipids), alcohols (e.g., polyalcohol), carbohydrates and other natural substances such as alginate. Further, it is contemplated that other materials may be added to the hollow fibers or coated on to the hollow fibers that aid in cell attachment and cell growth. For example, it is contemplated that the hollow fiber additive or coating is one or more of proteins, hydrogels, or other coatings known by one of skill in the art including extra cellular matrix (ECM) components and extracts, poly-D-lysine, laminin, collagen (e.g., collagen I and collagen IV), gelatin, fibronectin, plant-based ECM materials, collagen-like, fibronectin-like and laminin-like materials known to one of ordinary skill in the art that are isolated from a plant or synthesized from more simple substances. The overall result is that the fibers of the present invention impart the texture and structure of meat and meat products giving the structured clean meat product produced by the present invention a texture, appearance, handleability and mouth feel similar to real meat.

More specifically, the hollow fibers of the present invention may comprise one or more of cellulose, chitosan, collagen, zein, alginate, agar, inulin, gluten, pectin, legume protein, methyl cellulose(s), gelatin, tapioca, xanthan/guar/tara/bean/other gums, proteins (e.g., polypeptides, peptides, glycoprotein and amino acids including, but not limited to, various forms of corn/potato/rice/wheat/sorghum starches, plant isolates and soy/zein/casein/wheat protein, all of which are known to one of skill in the art), lipids, (for example, free fatty acids, triglycerides, natural waxes, and phospholipids). Cellulosic polymers may include cellulose acetate-butyrate, cellulose propionate, ethyl cellulose, methyl cellulose, nitrocellulose, etc. More specifically, the hollow fibers of the present invention may comprise a mixture of one or more legume proteins and hydrocolloids.

In an embodiment, it is contemplated that the hollow fibers of the present invention are edible and dissolvable or, edible or dissolvable. In other words, the fibers may be either edible or dissolvable or both. Further still, for fibers that are dissolvable, there may be differing degrees of dissolvability. For example, some fibers may be readily dissolvable upon exposure to a suitable solvent (e.g., a non-toxic solvent that is generally recognized as safe by the Food and Drug Administration (FDA) or other organization recognized as being qualified to assess the safety of consumable substances). Other fibers may be less readily dissolvable. In this regard, the less readily dissolvable fibers may be partly dissolved after the cells being cultured have reached the requisite level of confluency thereby leaving enough of the fiber to provide for a desired mouth feel and texture to the structured clean meat of the present invention but not an excess of fiber that may make the structured clean meat product of the present invention seem tough or chewy. Dissolvable hollow fiber constituents are known to those of skill in the art. For example, alginate is dissolvable upon exposure to a Ca²⁺ chelator. In an embodiment of the present invention, it is contemplated that the hollow fibers of the present invention comprise an amount of alginate to render the fibers partially dissolvable and/or a percentage of fibers in a device comprising the hollow fibers of the present invention comprise alginate.

Crosslinkers. In an embodiment of the present invention, it is contemplated that one or more crosslinkers are used in the hollow fibers of the present invention. Crosslinkers, as the name implies, bind one or more of the other constituents of the hollow fiber to strengthen the fiber. In an embodiment of the present invention, the crosslinker may be the dissolvable component or one of the dissolvable components of the hollow fibers of the present invention. Exemplary crosslinkers and crosslinking mechanisms as contemplated by the present invention, include but are not limited to, covalently bonded ester crosslinks (U.S. Pat. No. 7,247,191) and UV-crosslinking (U.S. Pat. No. 8,337,598), both of which are incorporated herein by reference in their entirety. Further, use of crosslinkers in the production of hollow fibers is known to one of skill in the art. See, for example, U.S. Pat. Nos. 9,718,031; 8,337,598; 7,247,191; 6,932,859 and 6,755,900, all of which are incorporated herein in their entirety.

Hollow fiber manufacturing techniques are known to one of skill in the art. (See, for example, Vandekar, V. D., Manufacturing of Hollow Fiber Membrane, Intl J Sci & Res, 2015, 4:9, pp. 1990-1994, and references cited therein). Known methods include, but are not limited to, melt spinning, dry spinning and wet spinning. In melt spinning, the polymer is heated to melting or above usually in an inert atmosphere. The melted polymer is then extruded though a “spinneret,” a nozzle sized to produce the desired size hollow fiber. The extruded polymer immediately solidifies and a capillary is formed with uniform structure and dimensions. The fibers may be further stretched to produce fibers with diameters less than 50 μm and a wall thickness as thin as 5 μm.

Dry spinning involves dissolving the polymer in a very volatile solvent. The solvent/polymer mixture is heated after extrusion and evaporation of the solvent the polymer solidifies.

Wet spinning is more versatile since the process involves a larger number of parameters that can be varied. The polymer and solvent mixture is extruded into a nonsolvent bath where demixing and/or phase separation occurs because of the exchange of solvent and nonsolvent. Between the extrusion and the nonsolvent bath there is an air gap where the hollow fiber membrane formation begins.

A technique that can eliminate or minimize the use of solvents is melt spinning with cold stretching (MSCS). This approach leads to cost effective production, but may sacrifice structure control and potential degradation of the food materials. In this technique the materials are heated for extrusion and then pulled as they are cooled as to mechanically form pores in the hollow fiber wall. All of three of these techniques have been widely studied and are known in the art they well summarized (see, Tan, X M. and Rodrigue, D., Polymers (Basel), 2019, Aug. 5:11 (8)).

Modifications of these techniques are also known to one of skill in the art. See, for example, WO 2011/108929 (incorporated herein by reference in its entirety) where a modified wet spinning extrusion process for the production of hollow fibers comprised of multiple polymers and polymer layers is disclosed. Manufacture of hollow fibers from non-synthetic materials is also known to one of skill in the art. See, for example, U.S. Pat. No. 4,824,569 to Suzuki, which is incorporated herein in its entirety.

The macroscopic structure of the hollow fibers of the present invention, in an embodiment, is contemplated to promote the orientation of the cells along the fibers. In this regard, it is desired by the present invention that the orientation of the component molecules from which the hollow fiber is constructed be oriented parallel, essentially parallel or predominately parallel to the length of the hollow fibers. In is further contemplated that the component molecules create a surface texture at least on the outer surface of the hollow fiber that aids in cell attachment and aids in cell orientation. Thus, in an embodiment, it is contemplated that the surface texture of the hollow fibers of the present invention create attachment points for cell attachment. In another embodiment, it is further contemplated that the cells grown on the hollow fibers of the present invention (in particular, the myocytes, myocyte-like cells or cells having characteristics of myocytes) orient and extend along the length of the hollow fiber similar to and resembling myocytes in vivo.

Thus, the orientation of the surface structure of the scaffold directly correlates to the alignment of the myotubes during formation. It can be thought of as if skeletal muscle wants to form along a preexisting structure. It can be envisioned that a bundle of fibers closely mimics skeletal muscle structure for the formation of aligned myotubes. Therefore, a hollow fiber bioreactor doesn't only achieve the tissue-like cell densities, but it also achieves the myotube alignment that other technologies do not, resulting in the most realistic mouth feel of all discussed technologies. The alignment phenomena can be better understood by reviewing: My mistake: Decellularized Apium graveolens Scaffold for Cell Culture and Guided Alignment of C2C12 Murine Myoblast—Santiago Campuzano, 2020, Ph.D. thesis, University or Ottawa, pp 58-59.

It is contemplated that the hollow fibers of the present invention have a range of sizes over which they will be suitable for the present invention. It is also contemplated that the hollow fibers of the present invention are spaced such that the cells grown on the hollow fibers achieve a density similar to that of real meat and with a minimum of void space between the cells. In one embodiment, it is contemplated that the hollow fibers of the present invention have an outer diameter of about 0.1 mm to about 3.0 mm, a porosity of about 0% porosity (making it diffusion based) to about 75%, and a wall thickness of about 0.008 to about 0.5 mm or about 0.01 mm to about 0.2 mm or any thickness between 0.008 mm to 0.5 mm not specifically iterated above. It was found by the present inventors that this size is suitable for the transport of media through the lumen of the fiber and permit the adequate flow of media through the wall of the hollow fiber while at the same time being rigid enough to support cell growth and, further, provide for the desired final product structure, texture, handleability and mouth feel. However, depending on the desired structured clean meat product (e.g., beef, poultry, fish, pork, etc.) other embodiments with regard to variations of the diameter, wall thickness and porosity of the fibers are contemplated; discussed infra.

Fiber porosity. The hollow fibers of the present invention need to have a porosity that allows for adequate flow of media though the wall of the fiber while at the same time ensuring a suitable surface for cell growth and cell support. The porosity of the hollow fibers is related, in part, to the thickness of the wall of the hollow fiber and to the composition of the hollow fiber. If the wall is thin enough, then 0% porosity may suffice allowing the media diffusing through the hollow fiber wall. The porosity of the hollow fibers of the present invention may be as high as 75%. Thus, the range of porosity of the hollow fibers of the present invention is from 0% to about 75%, from about 10% to about 65%, from about 30% to about 60%, or any percentage value between 0% and 75% not specifically iterated above.

The hollow fibers of the present invention may also be subject to a pore forming step. The pore forming mechanism will be one of the following techniques, well known in the art of membrane formation: IPS=thermally induced phase separation, NIPS=nonsolvent induced phase separation, VIPS=vapor-induced phase separation, MSCS=melt-spinning combined with stretching, (see, Review on Porous Polymeric Membrane Preparation. Part II: Production Techniques with Polyethylene, Polydimethylsiloxane, Polypropylene, Polyimide, and Polytetrafluoroethylene Xue Mei Tan 1, 2, 2019). In all scenarios the polymer will be in a liquid phase either by thermally melting it or chemical dissolution. From there, the polymer is extruded into a cylindrical shape, and drawn onto a spindle. During the extrusion step, a bore fluid can be used to prevent the hollow fiber form collapsing on itself. Between the extruding nozzle and the rewind spindle, there may also be a pore forming chamber, such as a water bath or an atmospheric environmental chamber.

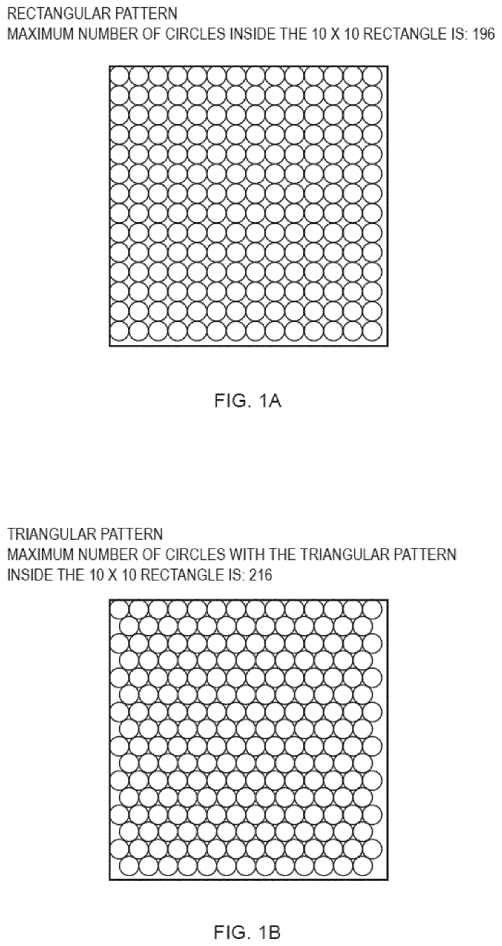

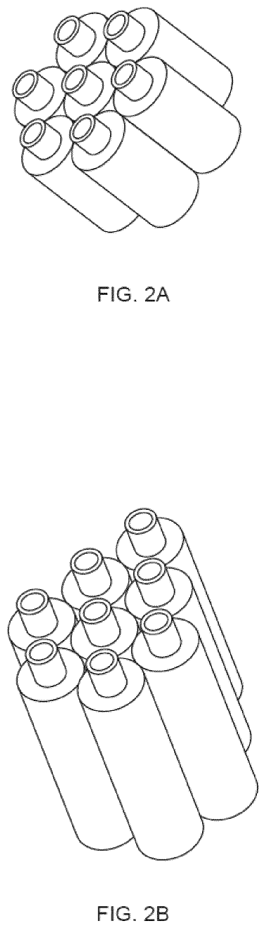

The present invention also contemplates the configuration of the hollow fibers of the present invention in a bioreactor. Fiber configuration may include one or both of fiber positioning and spacing. Fibers may be configured in any configuration that permits growth of the cell population with a minimum of void space between cells at confluency. For example, the fibers can be oriented in square/rectangle (rows and columns, see, FIG. 1A) or triangle/hexagonal (honeycomb, see FIG. 1B) packing modes. Other packing/spacing configurations are shown in FIG. 2. Thus, in one embodiment it is contemplated that the fibers are arranged such that the fibers, when viewed on end, form an ordered pattern of rows and columns. In another embodiment, it is contemplated that the fibers, when viewed on end, form a honeycomb pattern. In another embodiment, it is contemplated that the fibers of the present invention are arranged randomly or semi-randomly. In another embodiment, it is contemplated that the hollow fibers are arranged in an ordered or semi-ordered pattern of varying densities.

The hollow fibers can range from about 0.1 mm to about 3.0 mm, about 0.5 mm to about 2.0 mm and about 0.8 mm to about 1.3 mm in outer diameter, and any value in between the cited values. A 1.0 mm hollow fiber assumes about 0.3 mm to about 0.5 mm of meat growth around the outer diameter. An end diameter of approximately 1.1 mm can result in meat with about 85 hollow fibers/cm².

In another embodiment, it is contemplated that the fibers have varying degrees or amounts of space between fibers. For example, having rows of fibers at a higher density interspersed between fibers at a lower density may be used to produce changes in the texture of the final structured clean meat product, such as is common in natural fish meat. Further still, it is contemplated that fibers of varying diameters, porosities and wall thicknesses may be used in the same hollow fiber cartridge, again, to simulate the appearance, texture, handleability and mouth feel of natural meat.

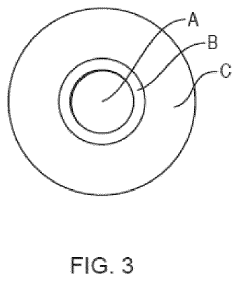

In any configuration, the fibers are spaced such that the spacing between the fibers is of a distance that permits an adequate flow of media (and the nutrients, growth factors, etc., contained therein) to reach all of the cell mass. This, of course, will be related at least in part on flow rate of the media and porosity of the hollow fiber walls but is related in greater part on physical distance from the surface of the outer wall of the hollow fiber to the cells. In other words, media and nutrients will only travel or defuse a limited distance through a cell mass. It is currently thought that the maximum for diffusion of oxygen and nutrients is 200 μm. Rouwkema, J., et al., (2009) Supply of Nutrients to Cells in Engineered Tissues, Biotechnology and Genetic Engineering Reviews, 26:1, 163-178. Thus, spacing between fibers should be about 400 μm from the outer wall of one fiber to the outer wall of a neighboring fiber. Without being limited to any specific dimensions, FIG. 2 shows representations of cell mass on a configuration of hollow fibers. Without being limited to any specific dimensions, FIG. 3 shows a representation of an embodiment of a cross section of a single hollow fiber of the present invention with A being the center or lumen of the hollow fiber, B being the porous hollow fiber wall and C being the cell mass. In culture conditions where media flows both through the hollow fibers and through the spacing between the hollow fibers the spacing can be greater. For example, spacing could be 800 μm from the outer wall of one fiber to the outer wall of a neighboring fiber. These figures are if the culture process relies on diffusion alone. However, use of a pump (for example) will create a flow of media from the hollow fibers, through the cell culture space between the hollow fibers and to the housing exits (rather than relying on diffusion alone) allowing the fibers to be spaced further apart. For example, in some embodiments it is contemplated the maximum distance between fibers is from about 0.05 mm (50 μm) to about 5.0 mm; about 0.1 mm to about 3.0 mm; about 0.1 mm to about 2.0 mm; about 0.1 mm to about 1.0 mm or about 0.2 mm to about 0.5 mm or any distance between the stated values. While it is a preferred embodiment that media flows from the center of the hollow fibers through the culture to the housing exits, it is also contemplated that the media flow can be in the reverse direction or can be alternated from one direction to the other, as desired. Alternating the direction of the media flow is believed to assist in ensuring all cells have an adequate media supply.

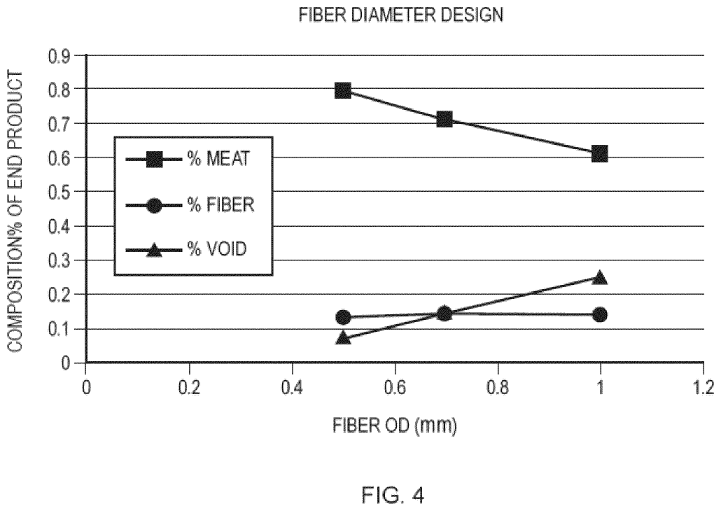

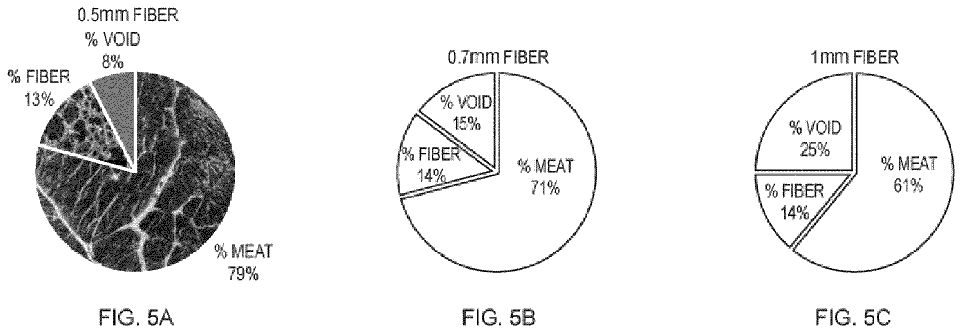

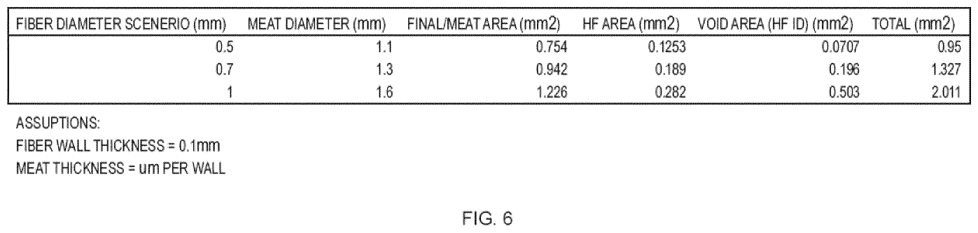

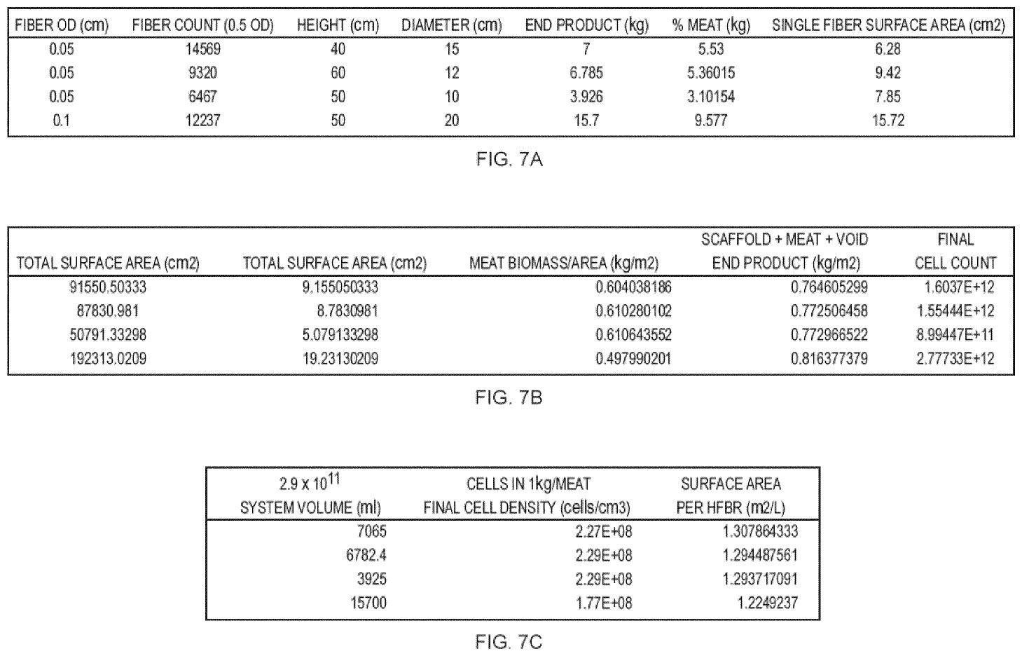

FIGS. 4-7 provide calculated data and schematic representations of the ratios between fiber, cells and void space that are acceptable for the present invention. FIG. 4 shows exemplary calculated percentages of clean meat (“meat”), fiber and void space based on different fiber diameters (OD=outer diameter). One of skill in the art will be able to extrapolate from these figures for fibers of larger or narrower ODs. FIG. 5 shows exemplary calculated percentages of clean meat (“meat”), fiber and void space for three different fiber ODs. FIG. 6 presents the exemplary data from FIG. 5 in table format. FIG. 7 shows exemplary calculated data on embodiments of the hollow fibers of the present invention in table format.

It is an embodiment of the present invention that a degree of randomness will be inherent in the distancing of the hollow fibers of the present invention. The figures given in the previous paragraph are average fiber-to-fiber distances for a given assembly. In an embodiment of the present invention, spacers and/or assembly techniques may be used to ensure, normalize or control the distances between the fibers. See, for example, Han G, Wang P, Chung T S., Highly robust thin-film composite pressure retarded osmosis (PRO) hollow fiber membranes with high power densities for renewable salinity-gradient energy generation, Environ Sci Technol. 2013 Jul. 16; 47(14):8070-7. Epub 2013 Jun. 28 or Chun Feng Wana, Bofan Li a, Tianshi Yang a, Tai-Shung Chung, Design and fabrication of inner-selective thin-film composite (TFC) hollow fiber modules for pressure retarded osmosis (PRO), Separation and Purification Technology, 172:32-42, 2017.

Once the cell density becomes too dense or the thickness of the cell mass becomes too thick, the ability of the media to reach the cells furthest away from the hollow fiber becomes difficult. A lack of media to these cells may result in dead cells in the reactor and/or dead spaces where cells cannot grow. The corollary is that the media needs to flow through the hollow fiber cartridge to the housing exits. That is, a flow of media needs to be maintained at least until confluency is reached and the structured clean meat product is harvested. One of skill in the art, based on the teachings of this specification, will be able to calculate the correct spacing of and porosity of the fibers of the present invention for a given desired structured clean meat product.

The hollow fibers of the present invention can be arranged and secured in what is referred to herein as a “hollow fiber cartridge.” In one embodiment, it is contemplated that the hollow fiber cartridge is made by having the ends of the hollow fibers are secured in an end piece in the desired arrangement. For example, each fiber has a first end and a second end. Each end is secured in an end piece, that is, a first and a second end piece. An end piece can be, for example, a resin or plastic that is known in the art to be inert and non-toxic to cells. At least one of the first or second ends of the hollow fibers is positioned in the end piece such that the interior lumen of the hollow fiber is in fluid communication with the exterior environment. Thus, with this positioning of the hollow fibers in the end piece, media can be caused to flow from the exterior environment of the hollow fiber (i.e., outside of the hollow fiber but inside of, for example, a sterile bioreactor) into the inner lumen of the hollow fiber.

One of skill in the art understands how to assemble hollow fibers into a module or cartridge. These techniques are applicable to the hollow fibers of the present invention. In brief, after spinning, the hollow fibers are cut to length and the ends of the fibers encased (i.e., potted) in a resin that will flow around the fiber ends and solidify. Sometimes, the section of the fibers may be encased in a substance (e.g., Plaster of Paris or other easily removable material known to one of skill in the art) to close the pores of the fibers so that the “potting solution,” i.e., the liquid resin, does not enter or plug the pores in the fibers. See, for example, Vandekar, V. D., Manufacturing of Hollow Fiber Membrane, Intl J Sci & Res, 2015, 4:9, pp. 1990-1994, and references cited therein. In the present invention, one or both of the ends of the “potted” bundle are trimmed or cut to expose the open ends of the fibers to permit the flow of media once the bundle is inserted into a housing for use in the production of the structured clean meat of the present invention.

Further still, it is contemplated in some embodiments that the hollow fiber cartridge of the present invention has securing devices to maintain a desired distance between the first and second end piece. This may be necessary or preferred, for example, for easier insertion of the hollow fiber cartridge of the present invention into, e.g., a bioreactor housing.

Thus, it is contemplated that in one embodiment the hollow fiber cartridge of the present invention contains a plethora of hollow fibers arranged in a desired arrangement. The hollow fibers of the present invention have a first end and a second end. The arrangement is maintained by securing the first end and the second end of the hollow fibers in a first and a second end piece. The hollow fibers, once secured as describe, are then positioned parallel, substantially parallel or essentially parallel to each other. Further, the first and second end pieces are positioned parallel, substantially parallel or essentially parallel to each other. Further still, the hollow fibers of the hollow fiber cartridge of the present invention are positioned perpendicular, substantially perpendicular or essentially perpendicular to the end pieces of the hollow fiber cartridge of the present invention. The diameter and length of the hollow fiber cartridge will depend on the desired structured clean meat product being produced and bioreactor configurations.

In an embodiment of the present invention, it is contemplated that the hollow fibers of the hollow fiber cartridge of the present invention are at an average density of about 40-about 120 per cm², at an average density of about 60-about 100 per cm², at an average density of about 70-about 90 per cm² or any value between the values given above but not specifically iterated.

In an embodiment of the present invention, it is contemplated that the hollow fibers in the hollow fiber cartridge of the present invention have a void space between the hollow fibers prior to the addition of cells and, the void space between the hollow fibers is about 25%-about 75% of the total area of the hollow fiber cartridge or about 40%-about 60% of the total area of the hollow fiber cartridge or any value between the values given above but not specifically iterated.

In an embodiment of the present invention, it is contemplated that the hollow fiber cartridge of the present invention is designed to be removably inserted into a housing. That is, the cartridge can be inserted into the housing at the beginning of a production run and removed, i.e., harvested, at the end of the production run for any further desired processing of the structured clean meat product of the present invention. After harvesting of the structured clean meat product, a new hollow fiber cartridge of the present invention may be inserted into the housing and the process repeated. In this regard, the housing for the hollow fiber cartridge of the present invention is part of a bioreactor or bioreactor system.

Reactor configuration. The present invention is not limited to any particular reactor configuration or reactor system configuration so long as adequate media flow can be maintained through the culture and waste products removed. Hollow fiber reactors are typically tubular in shape although they can be oval, flat (sheet-like), rectangular or any other shape. In a preferred embodiment, the reactor comprises an insertable/removable insert that comprises the hollow fibers of the present invention. After confluent cell growth (as defined herein) is reached the insert can be removed and product finalized by removal of the insert ends and any further desired processing. Further processing may take the form of, for example, slicing, surface texturing, adding flavors, etc. Alternatively, further meat enhancement can take place before the harvest and disassembly of the device. For example, the media can be flushed out of the hollow fiber device and then the additives would be pumped directly into or around the fibers.

Non-limiting examples of suitable reactor systems. The most suitable type of reactor system is the feed batch system although it is contemplated that any available reactor will be suitable for use with the hollow fibers and hollow fiber cartridge of the present invention. For example, the Mobius® system (MilliporeSigma, Burlington, MA) is an example of a commercial system that can easily be converted to use with the present invention. The bioreactor in which the structured clean meat product is produced (i.e., the reactor comprising the hollow fibers of the present invention) may be seeded with cells grown in another bioreactor. The bioreactor that is seeding the hollow fiber device (a reactor suitable for cell growth (proliferation) and cell expansion) can be an existing commercial reactor, for example, a stirred tank or wave-type reactor. The proliferation/expansion bioreactor is contemplated to be, for example, a stirred tank or wave-type reactor (as are known to one of ordinary skill in the art) and to be a suspension, agglomerated biomass, microcarrier culture, or other suitable reactor known to one of ordinary skill in the art. It is contemplated that the production bioreactor (i.e., the reactor comprising the hollow fibers of the present invention) may be, for example, single use, multi-use, semi-continuous or continuous. The present invention further contemplates a manifold of multiple reactors comprising the hollow fiber of the present invention.

Thus, it is contemplated that an exemplary reactor system of the present invention comprises one of more hollow fiber cartridges of the present invention, a housing sized to hold said hollow fiber cartridge; a medium source fluidly connected to one or more inlets in said housing; one or more medium outlets in said housing; and, one or more pumps to supply the medium to and/or remove waste medium from said hollow fiber cartridge through said medium inlet(s) and/or outlet(s). Further still, the inlets are fluidly connected to the interior of the hollow fibers. Yet further still, the hollow fiber bioreactor may comprise an automated controller or automatically controlled system.

The present invention also contemplates a process for producing a meat product, comprising; seeding a void space between the hollow fibers in a hollow fiber reactor of the present invention with one or more of myocytes, myocyte-like cells or engineered cells expressing one or more myocyte-like characteristics at a density of, for example, 100,000 cells to 100,000,000 (10⁵-10⁸) (Radisic, et al., Biotechnol Bioeng, 2003 May 20:82(4):403-414.) and culturing the cells until achieving about 80%-about 99% confluency, 85%-about 99% confluency, about 90%-about 99% confluency, about 95%-about 99% confluency, about 98%-about 99% confluency or about 100% confluency (or any value in between the recited percent values), removing said first holding device and said second holding device from the first ends and second ends, respectively, of said hollow fibers.

After seeding, the hollow fiber cartridge has media supplied to the cells through one or both of the first end and second end of the hollow fibers into the interior of the hollow fibers, through the wall of the hollow fibers into the void space between the hollow fibers where said cells are seeded and through one or more of said outlets in said housing. In another embodiment, it is contemplated that media can also flow between fibers from both the inlet(s) and outlet(s) of device. For example, one fluid path is through fiber wall and the second fluid path is around the fibers. It is contemplated that the device may have multiple inlets and outlets. After the cells achieve confluency, flushing out any residual media and waste products and infusing the interior of the hollow fibers and/or any remaining void space between the cells with one or more of fats, flavors, colors, salts and preservatives.

Fats suitable for addition to the structured clean meat product of the present invention include, but are not limited to: saturated, monounsaturated, polyunsaturated fats such as corn oil, canola oil, sunflower oil, and safflower oil, olive oil, peanut oil, soy bean, flax seed oil, sesame oil, canola oil, avocado oil, seed oils, nut oil, safflower and sunflower oils, palm oil, coconut oil, Omega-3, fish oil(s), lard, butter, processed animal fat, adipose tissue, or cellular agriculture derived fat, or combinations thereof. Synthetic fats such as oleoresin may also be used. In fact, any fat recognized by the Food and Drug Administration (FDA) is suitable for use in the present invention and contemplated for use in the structured clean meat product of the present invention. On the FDAs food additive list, natural substances and extractives (NAT), Nutrient (NUTR), Essential oil and/or oleoresin (solvent free) (ESO).

Flavors suitable for use in the structured clean meat product of the present invention include, but are not limited to, any flavor documented on the FDA's food additive list. These may be documented as natural flavoring agents (FLAN), essential oils and/or oleoresin (solvent fee) (ESO), enzymes (ENZ), natural substances and extractives (NAT), non-nutritive sweetener (NNS), nutritive sweetener (NUTRS), spices, other natural seasonings & flavorings (SP), synthetic flavor (SY/FL), fumigant (FUM), artificial sweeteners including aspartame, sucralose, saccharin and acesulfame potassium and yeast extract, or combinations thereof, are contemplated for use in the structured clean meat product of the present invention.

Texture Enhancers suitable for use in the structured clean meat product of the present invention include, but are not limited to, pureed plant material, guar gum, cellulose, hemicellulose, lignin, beta glucans, soy, wheat, maize or rice isolates and beet fiber, pea fiber, bamboo fiber, plant derived fiber, plant derived gluten, carrageenan, xanthan gum, lectithin, pectin, agar, alginate, and other natural polysaccharides, grain husk, calcium citrate, calcium phosphates, calcium sulfate, magnesium sulfate and salts, or any combination thereof, are contemplated for use in the structured clean meat product of the present invention. These may be documented on the FDA's food additive list as solubilizing and dispersing agents (SDA), and natural substances and extractives (NAT).

Nutritional Additives suitable for use in the structured clean meat product of the present invention include, but are not limited to, vitamins, trace elements, bioactive compounds, endogenous antioxidants such as A, B-complex, C, D, E vitamins, zinc, thiamin, riboflavin, selenium, iron, niacin, potassium, phosphorus, omega-3, omega-6, fatty acids, magnesium, protein and protein extracts, amino acids salt, creatine, taurine, carnitine, carnosine, ubiquinone, glutathione, choline, glutathione, lipoic acid, spermine, anserine, linoleic acid, pantothenic acid, cholesterol, Retinol, folic acid, dietary fiber, amino acids, and combinations thereof, are contemplated for use in the structured clean meat product of the present invention. Any food additive or additives that are generally recognized as safe (GRAS) or approved by the FDA are contemplated for use in the structured clean meat product of the present invention and incorporated herein. See, for example: www.fda.gov/food/food-additives-petitions/food-additive-status-list.

Any food coloring or colorings, natural or artificial, that are Generally Recognized As Safe (GRAS) or approved by the FDA are contemplated for use in the structured clean meat product of the present invention. See, for example: www.fda.gov/industry/color-additive-inventories/color-additive-status-list.

Prophetic cell types. The hollow fibers of the present invention are designed to be used to grow specific cell types suitable for the production of in vitro or lab grown meat and meat products, i.e., the structured clean meat of the present invention. Therefore, while many different types of cells can grow on the hollow fibers (and in the hollow fiber cartridges of the present invention, if desired), the fibers were developed to be used to grow muscle cells (i.e., myocytes), or cells with the characteristics of muscle cells or engineered to have the characteristics of muscle cells (collectively referred to herein as muscle cells or myocytes), to confluency and to mimic the natural structure of muscle (i.e., meat). Preferably, the muscle is skeletal muscle. That is, the hollow fibers of the present invention are designed by the inventors to be suitable to grow myocytes to obtain muscle fibers or myofibrils. Further, other types of cells may be grown on the hollow fibers of the present invention and in reactors comprising the hollow fibers of the present invention. These cells may be grown independently or in combination with muscle cells. For example, adipocytes or cells having the characteristics of adipocytes or engineered to have the characteristics of adipocytes (collectively referred to herein as adipocytes) may be cultured with the muscle cells to achieve an end product resembling natural muscle or meat. The hollow fibers of the present invention are also suitable for including other cells to be co-cultured with the muscle cells of the present invention, for example, fibroblasts, cells having the characteristics of fibroblasts or cells engineered to have the characteristics of fibroblasts.

With specific regards to a co-culture of muscle cells and adipocytes, the ratio of muscle cells to adipocytes may be 99:1, 95:5, 92:8, 90:10, 88:12, 85:15 82:18, 80:20, 75:25 or any ratio from 100:0 to 75:25, inclusive.

The cells that are suitable for use with the present invention may be obtained from or derived from any animal from which food is now obtained. Prominent examples are bovine, porcine, ovine, piscine (e.g., fish such as tuna, salmon, cod, haddock, shark, etc.), shellfish, avian (e.g., chicken, turkey, duck, etc.). More exotic sources of cells may also be used, such as from animals that are traditionally hunted rather than farmed (e.g., deer, elk, moose, bear, rabbit, quail, wild turkey, etc.) or combinations thereof.

Cells used in the present invention may be derived by any manner suitable for the generation of differentiated cells having the characteristics desired. For example, any procedure suitable for deriving cells with differentiated myocyte-like characteristics, adipocyte-like characteristics, etc. Such characteristics for myocytes include, for example, but not necessarily limited to, having an appearance of a long, tubular cell and with large complements of myosin and actin. Myocytes also have the ability to fuse with other myocytes to form myofibrils, the unit of muscle that helps to give muscle, i.e., meat, its distinctive texture. Such characteristics for adipocytes (also referred to in the art as lipocytes and fat cells) include, for example, but not necessarily limited to, having large lipid vacuoles that may take up as much as 90% or more of the volume of the cell. The hollow fibers of the present invention provide, at least in part, a replacement of the connective tissue (referred to as “fascia” in the art) typically found in skeletal muscle.

Cells useful in the present invention include, but are not limited to, cells that are derived from mesenchymal stem cells or induced pluripotent stem cells (iPSC). iPSCs are cells engineered to revert to their pluripotent state from which numerous cells types can be derived. In other words, iPSCs are pluripotent stem cells that can be generated directly from a somatic cell. The technology was first reported in 2006 (Takahashi K, Yamanaka S, 25 August 2006, “Induction of pluripotent stem cells from mouse embryonic and adult fibroblast cultures by defined factors” Cell, 126 (4): 663-76), has advanced from that point on (see, for example: Li, et al., 30 Apr. 2014, “Generation of pluripotent stem cells via protein transduction” Int. J. Dev. Biol., 58: 21-27), includes the generation of muscle cells (see, for example: Rao, et al., 9 Jan. 2018, “Engineering human pluripotent stem cells into a functional skeletal muscle tissue” Nat Commun., 9 (1): 1-12) and is well known to one of ordinary skill in the art.

Unless defined otherwise, all technical and scientific terms used herein have the same meaning as commonly understood by one of ordinary skill in the art to which this invention pertains.

When introducing elements of the present disclosure or the preferred embodiments(s) thereof, the articles “a,” “an,” “the” and “said” are intended to mean that there are one or more of the elements. The terms “comprising,” “including” and “having” are intended to be inclusive and mean that there may be additional elements other than the listed elements.

The transitional phrases “comprising,” “consisting essentially of” and “consisting of” have the meanings as given in MPEP 2111.03 (Manual of Patent Examining Procedure; United States Patent and Trademark Office). Any claims using the transitional phrase “consisting essentially of” will be understood as reciting only essential elements of the invention and any other elements recited in dependent claims are understood to be non-essential to the invention recited in the claim from which they depend.

All ranges include all values within the cited range including all whole, fractional and decimal numbers, inclusive.

Exemplification

### Example 1A

Prophetic Exemplification of Production of the Hollow Fibers of the Present Invention.

A blend of zein, gluten and alginate are mixed into a solvent blend of glycerol and Ethanol. This viscous mixture is then extruded through a heated spinneret that has an inner diameter of 0.5 μm. Upon exiting the nozzle, the solvent rich fiber is submerged in a bath of water, completing the phase separation of the hollow fiber. The tension on the fiber during the rewind results in the pulling effect further structures the fiber. The spool of fiber is then cut where the fibers are in a parallel orientation, creating a bundle. From there the bundle is then inserted into the cartridge, potted, then cut. An additional endcapping step completes the fluid path through the lumen.

The devices that contain the hollows could be in either cylindrical (cartridge) format or rectangular (cassette) format. In both configurations the hollow fiber will oriented as desired before the potting step is initiated. The hollow fibers may be wrapped around a core to help the flow of media around the fibers. Alternative to a core component, a diffusion barrier can be used between the exterior housing and the hollow fibers. The potting material can be the same material as the hollow fiber or another GRAS approved material; or alternatively, a food grade synthetic polymer. The solidification of the potting material can be thermosetting or a thermoplastic. There will be some cutting procedure to expose all open ends of the hollow fibers before an endcap is bonded to the housing. The endcaps will have an inlet/outlet to the lumen of the fiber. The inlet/outlet for the fluid path around the fibers may be integrated into the housing or into the endcap.

### Example 1B

Exemplification of Production of Exemplary Hollow Fibers of the Present Invention

Zein's solubility in ethanol makes it an interesting material from this application. Unfortunately, it will work poorly in extrusion-based phase separation techniques due to its partial solubility in a water bath. The present inventors contemplated a system where zein was one component and a secondary polymer such as alginate, chitosan, or cellulose was used to aid in setting up a 3-dimensional structure. However, blending of these hydrophilic and hydrophobic polymers was difficult.

Alginate can be precipitated out with ethanol. The present inventors found that the 2% alginate starts to gel at around 25% ethanol. The present inventors contemplated a system where alcohol was included into either the coagulation bath or the polymer dope itself believing that would aid in the pore forming behavior, likely increasing the pore size of the end product.

Alginate & Protein System:

What made this system so surprising and unexpected was the ability to mix in high quantities of protein content and still be able to instantaneously solidify the hollow fiber structure without the use of harsh solvents, acid, or bases.

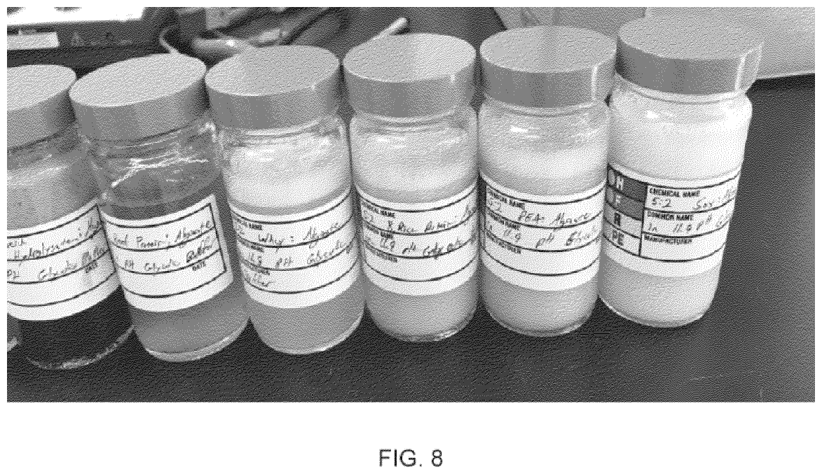

The present inventors made mixes by weighing out dry component of 2 g sodium alginate, 7 g protein powder and 91 g of water or buffer with 15 g/L calcium chloride as a crosslinker. Resulting in a solids ratio of 2:7 Alginate:protein. Ratios of Alginate:protein were also made from 2:0, 2:2, 2:4, 2:5, 2:7, 2:9. The system can be pushed to even higher protein ratios and the protein can be of plant or animal origin. As shown in FIG. 8, the protein samples exemplified here were (in order from left to right) soy acid hydrolysate, beef protein isolate, whey protein isolate, brown rice protein isolate, pea protein isolate, soy protein isolate. While not shown, we have also experimented with pumpkin protein isolate, sunflower seed protein isolate, along with others.

These combinations were mixed via homogenizer followed by hybridizer at 40° C. overnight. It was observed that the animal derived proteins have dissolution potential from the level of transparency seen. This is likely due to the non-soluble components in the plant-protein isolates (usually less than 10% of the solids).

These protein isolates were extruded through a coaxial needle sourced from the rame-hart instrument co. (Succasunna, NJ) via a syringe pump. The solidification bath used was MilliQ water (MilliporeSigma, Bedford, MA) with 15 g/L calcium chloride. After sitting in the calcium bath for several minutes, the fibers are removed and rinsed in MilliQ water. Once rinsed, they were submerged within a jar of MilliQ water and autoclaved at 126° C. for an hour.

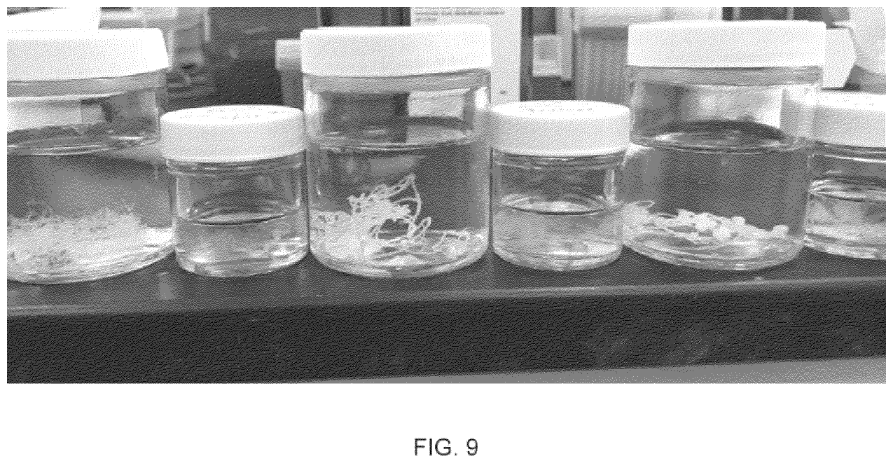

The present inventors next used alginate fibers without protein content (i.e., protein free) to demonstrate if there was an effect from the thermal treatment/autoclave. The fibers were 100% alginate for the solid content. The larger jars shown in FIG. 9 were autoclaved, where the smaller jars were not. Since the autoclaved fibers turn white, it can be concluded that the light no longer passes through them, indicating that there is a structural change of the polymer chains.

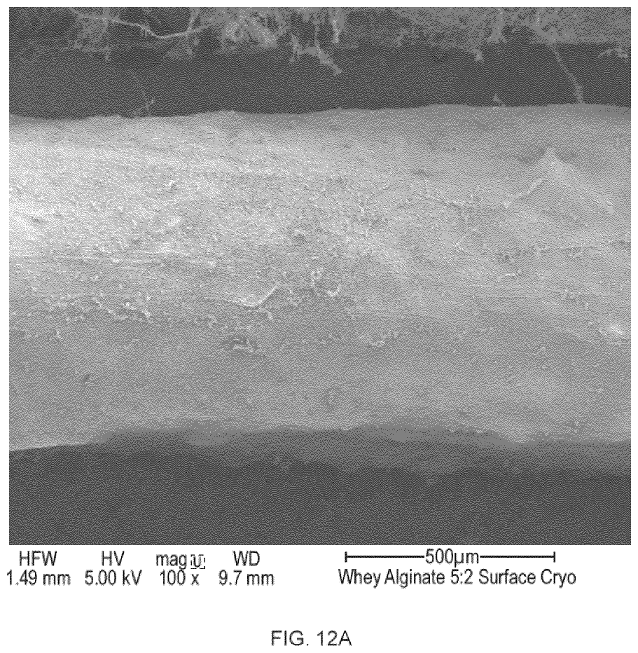

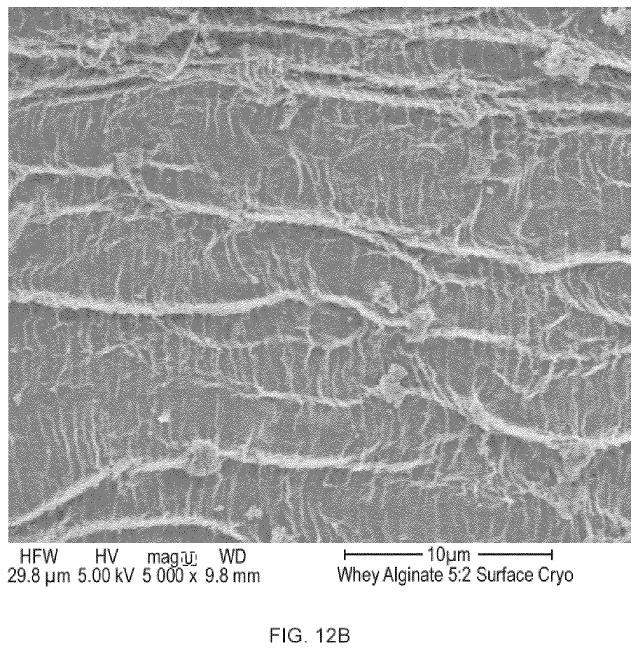

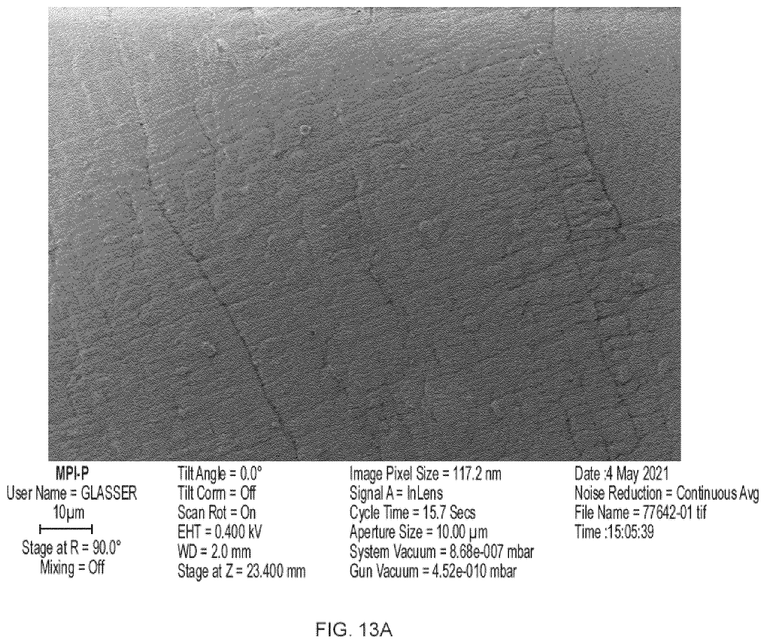

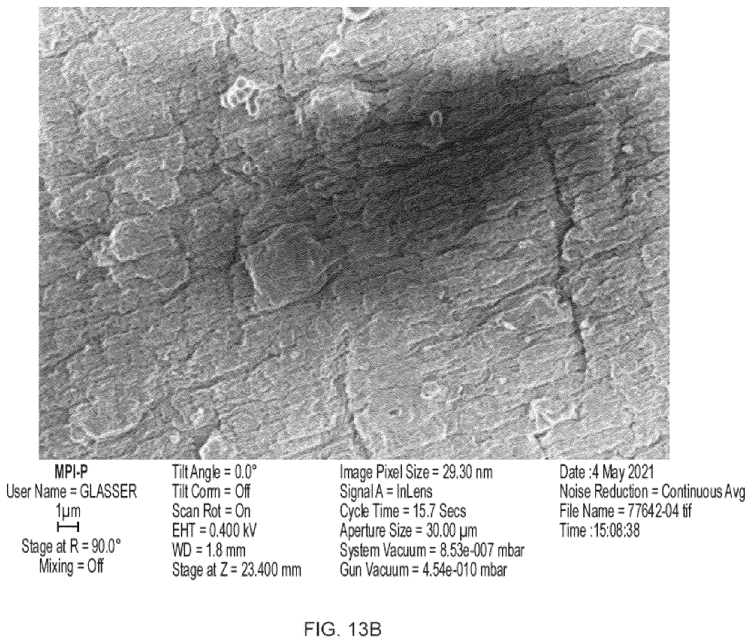

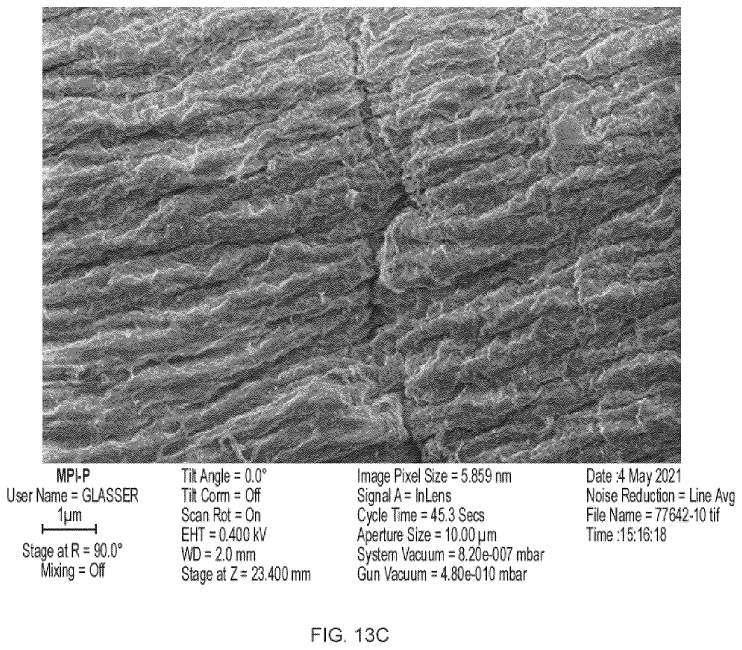

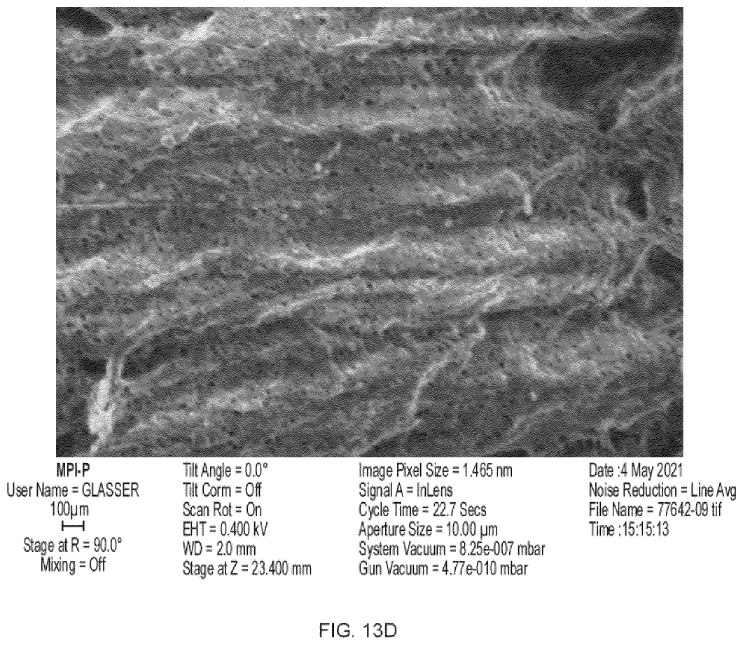

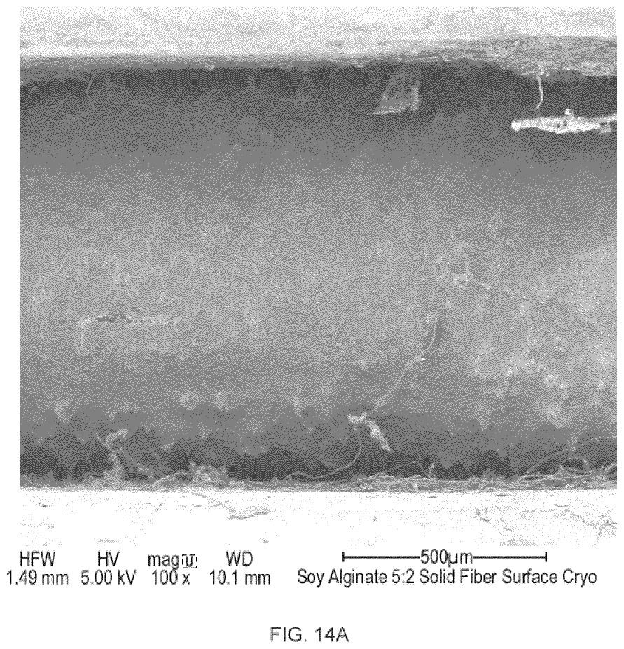

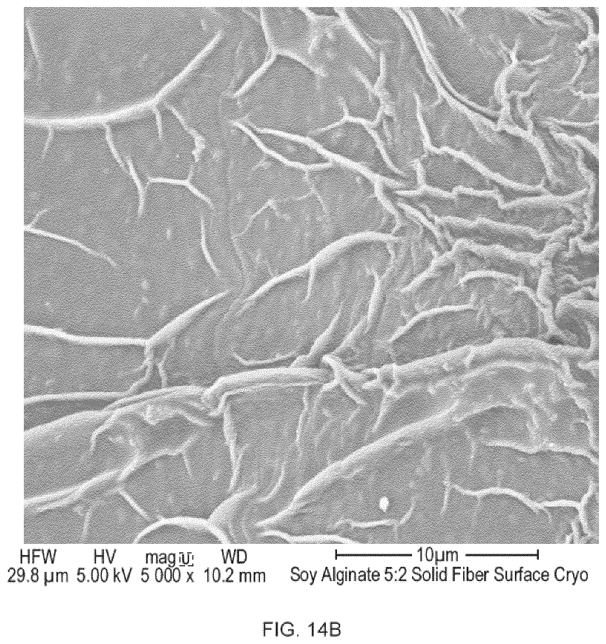

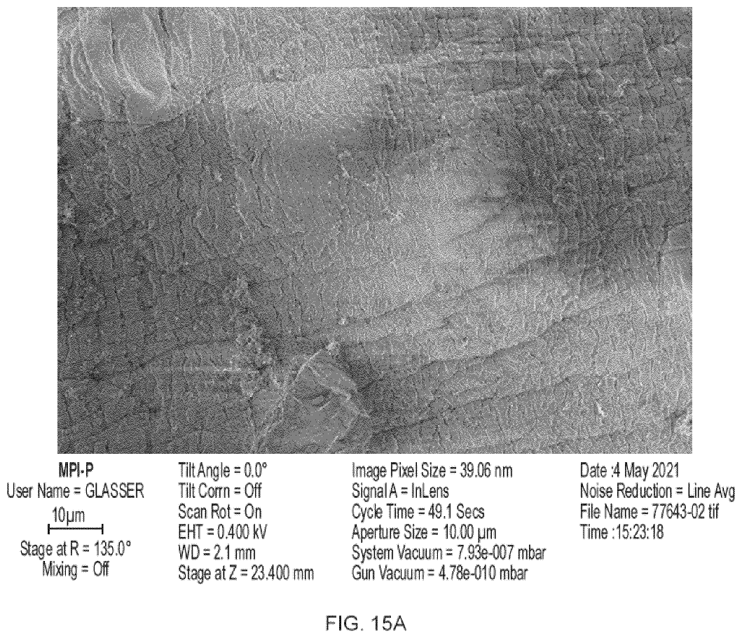

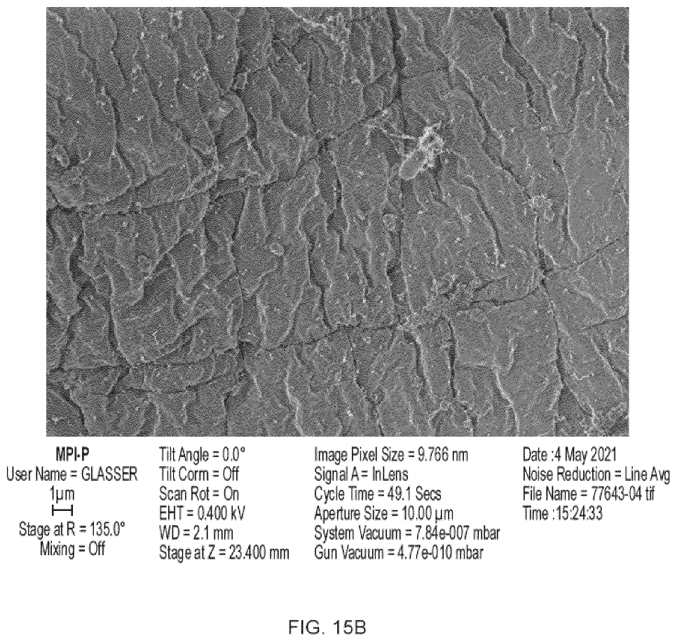

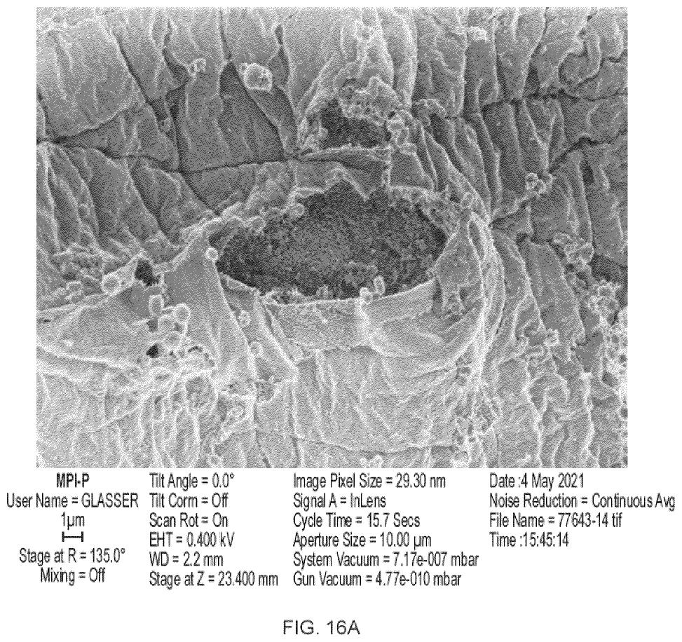

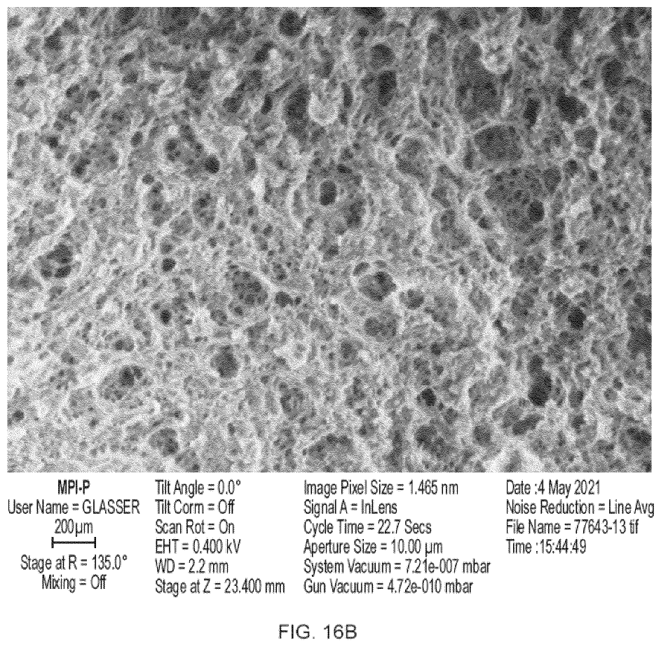

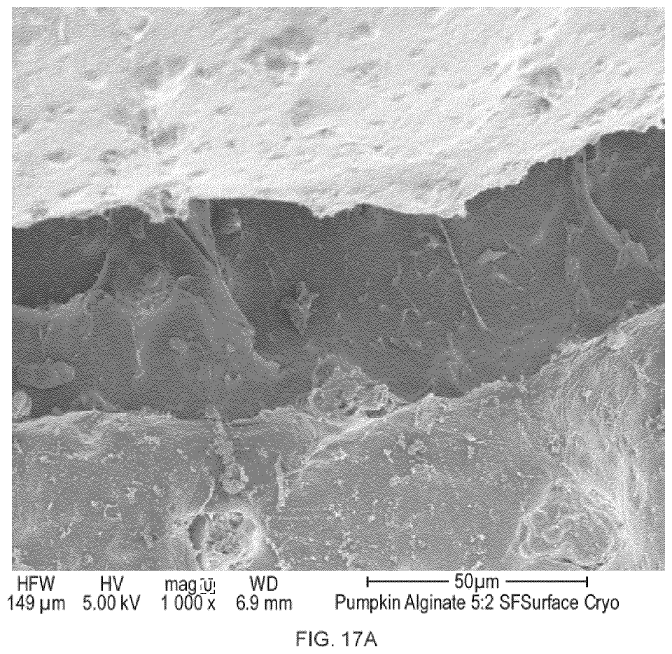

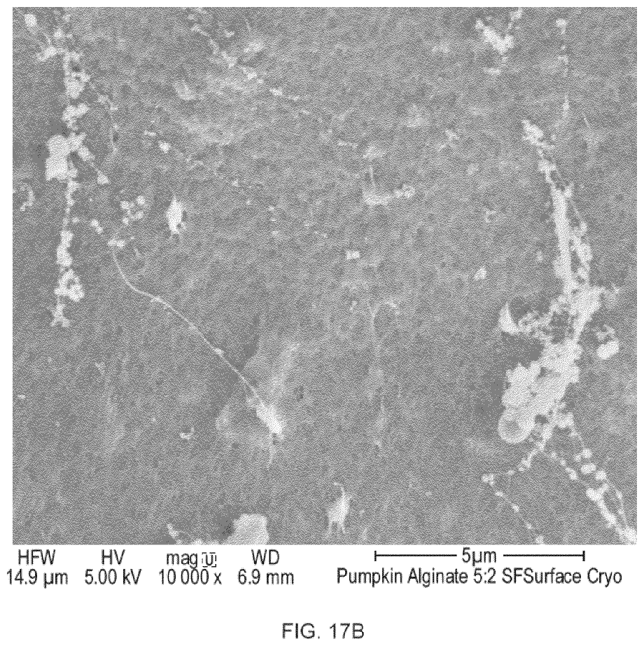

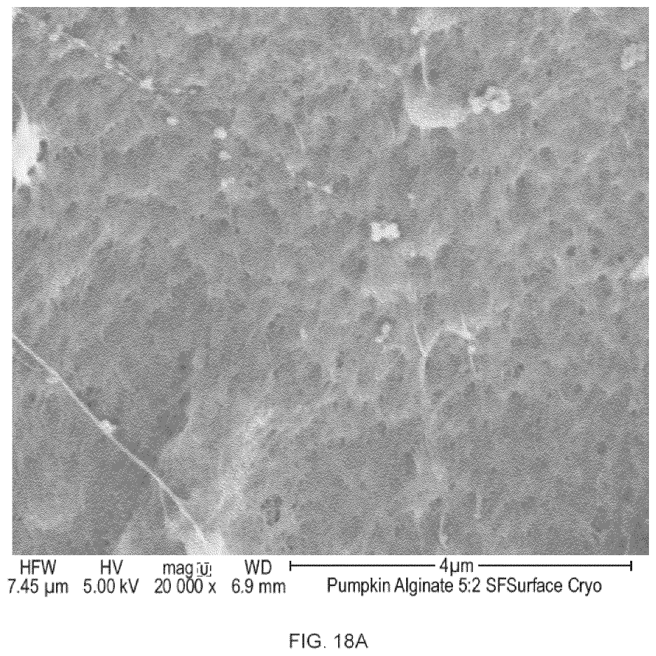

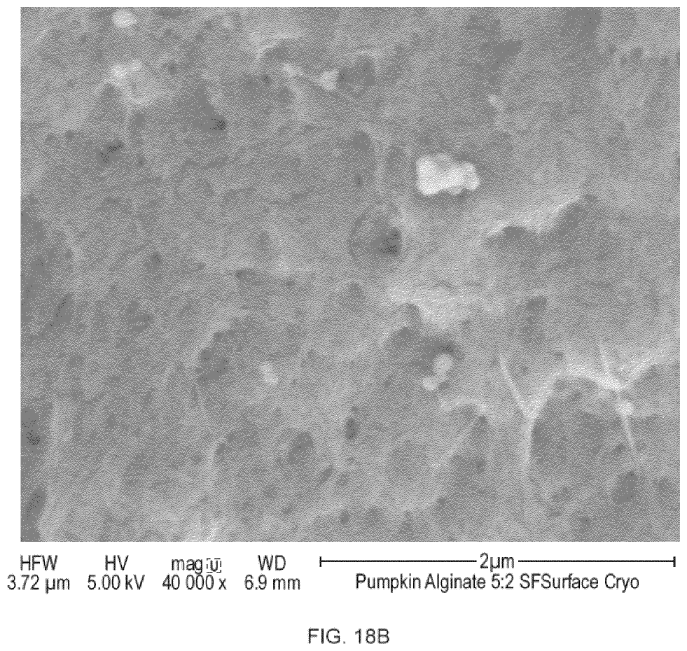

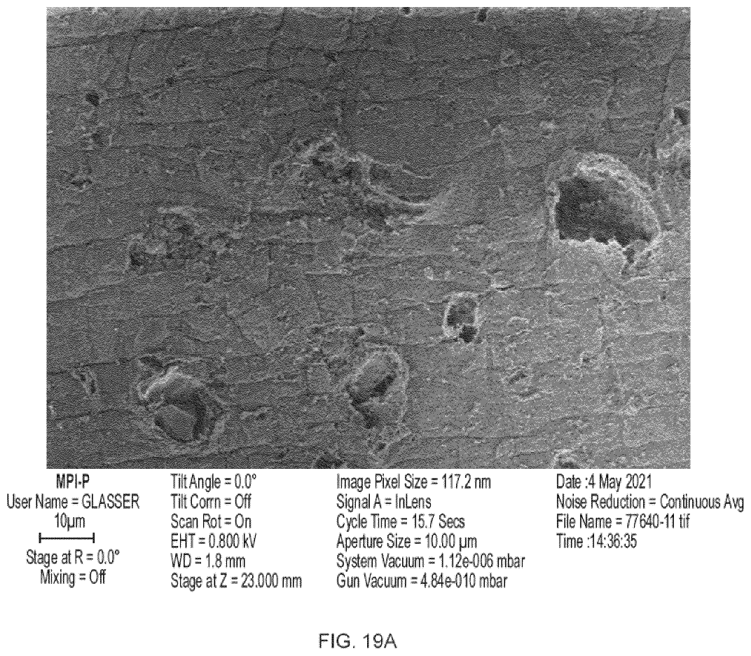

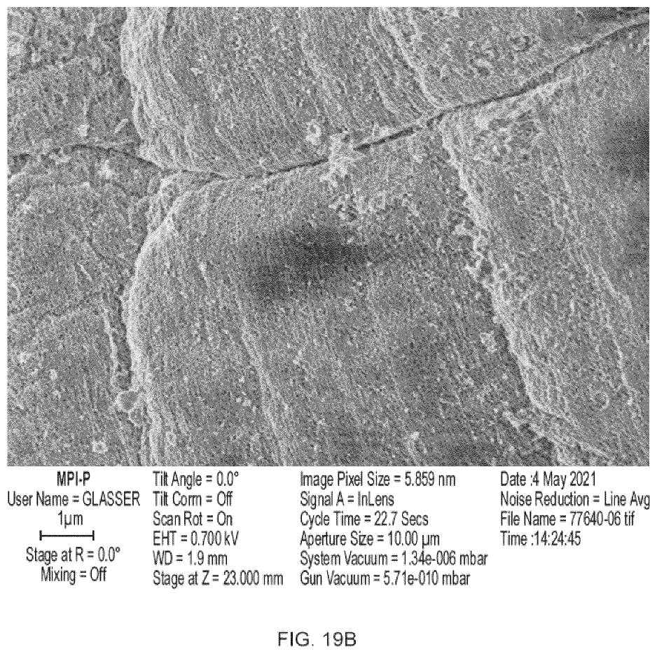

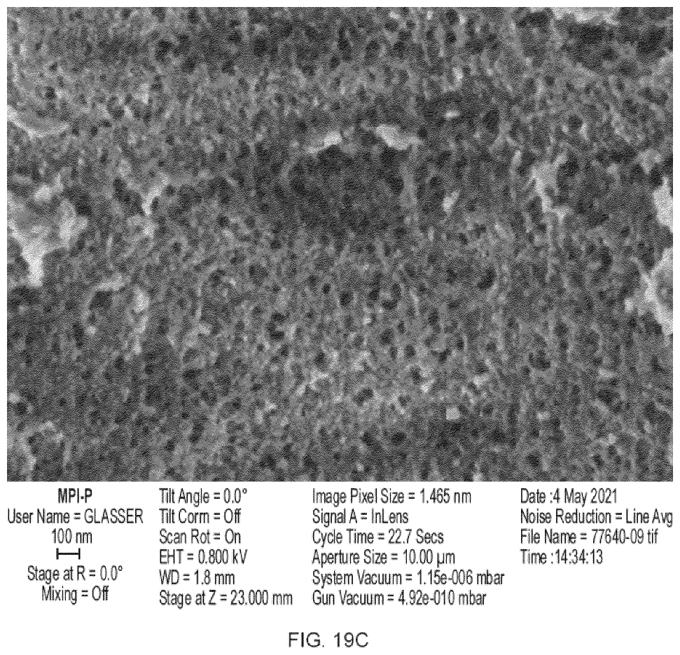

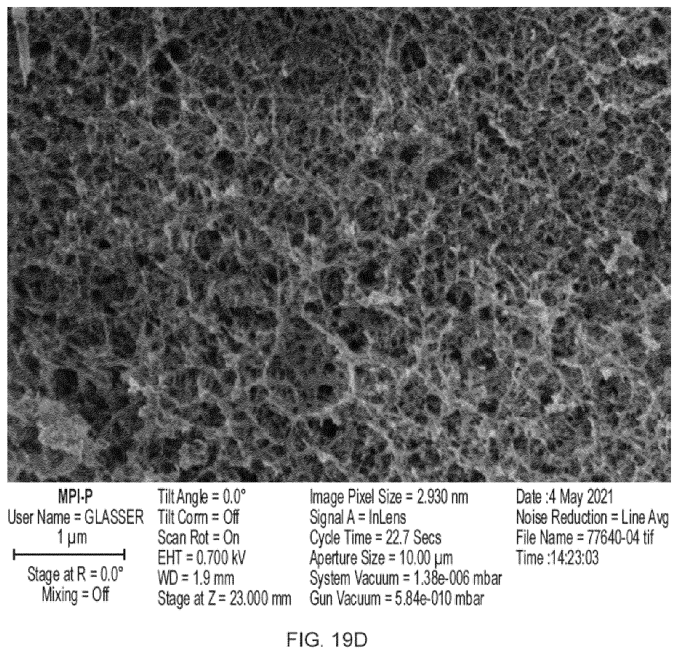

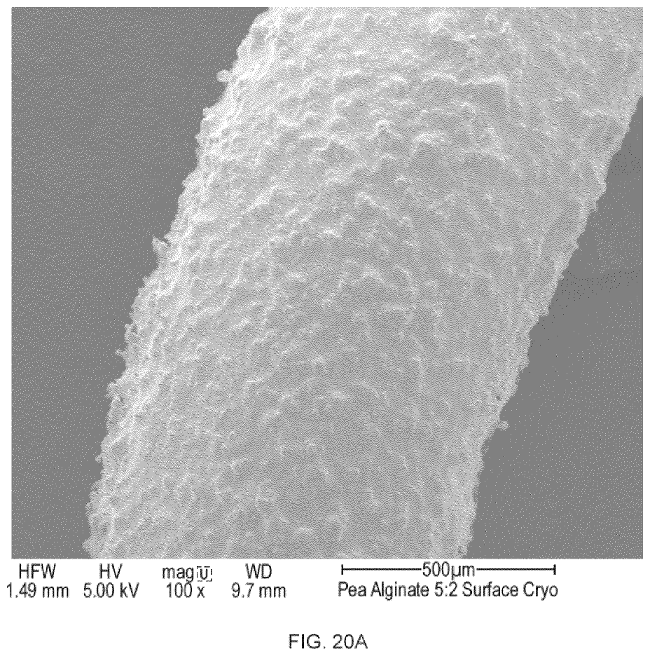

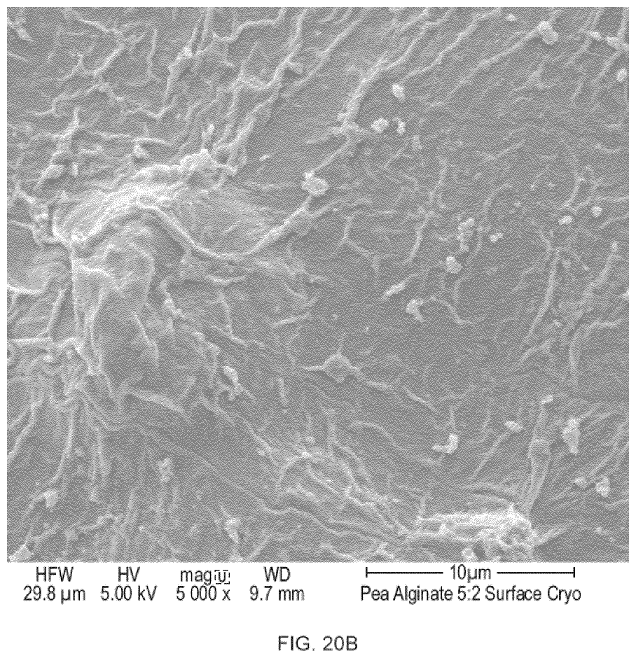

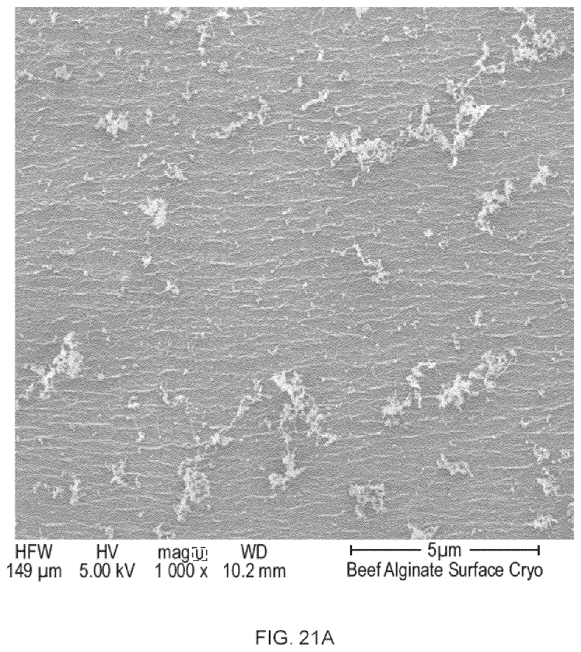

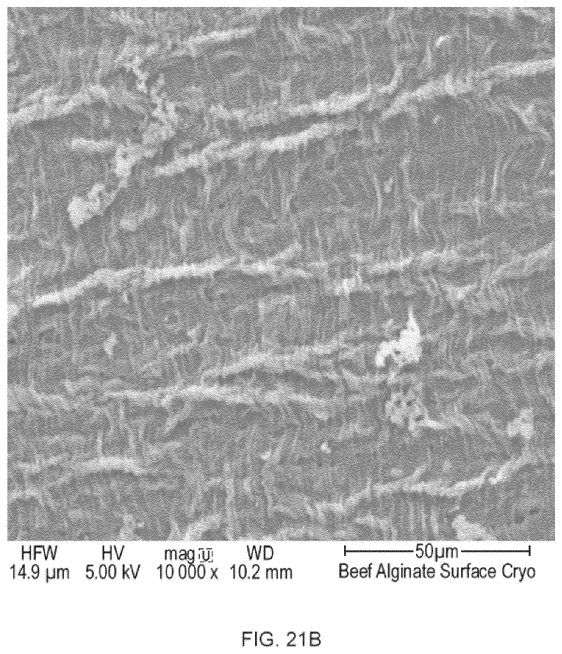

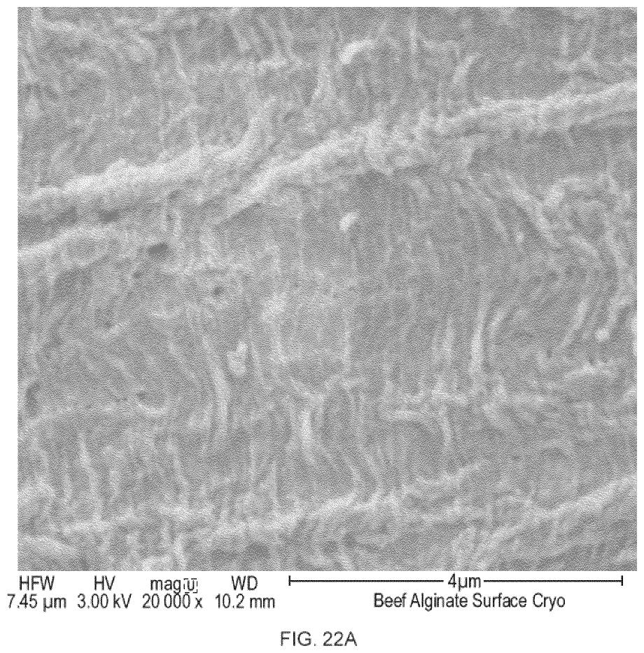

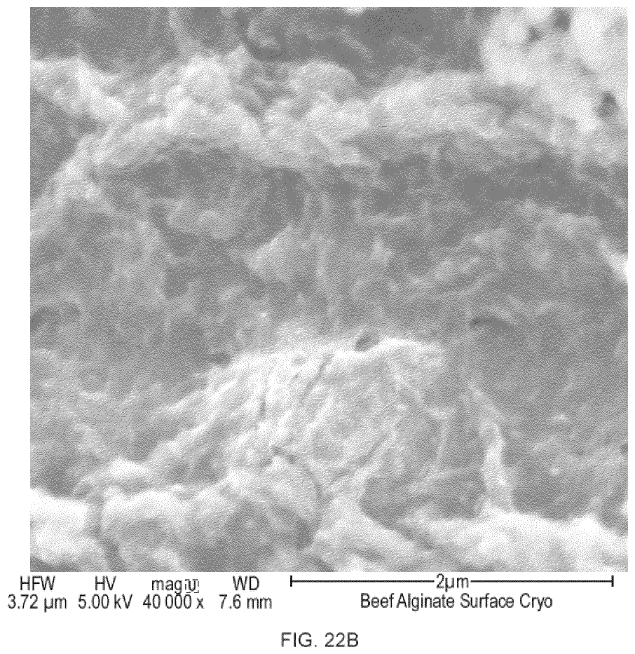

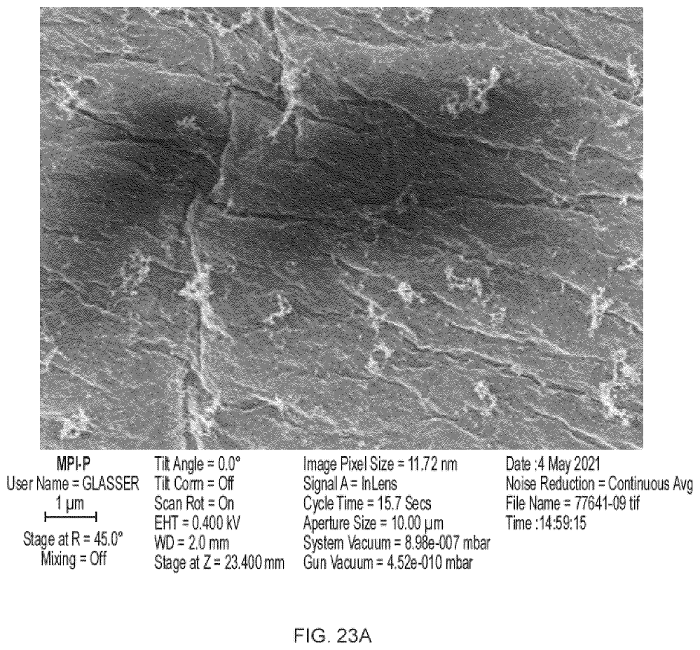

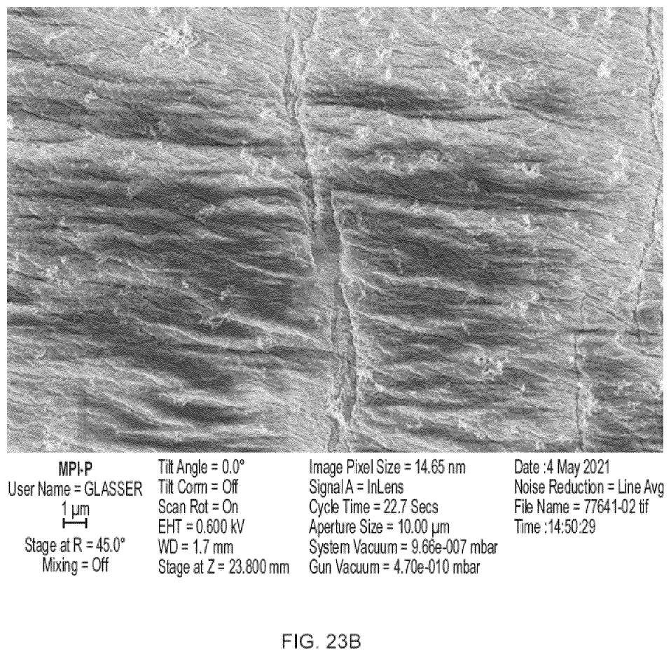

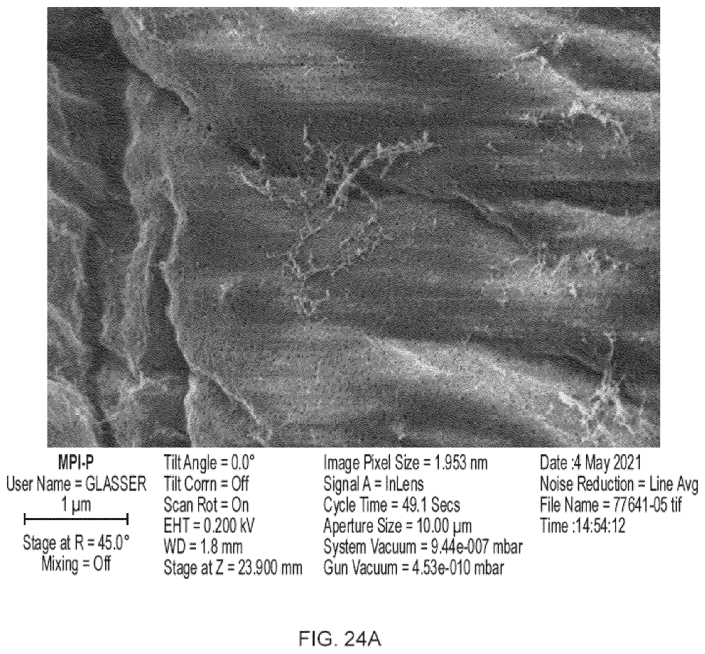

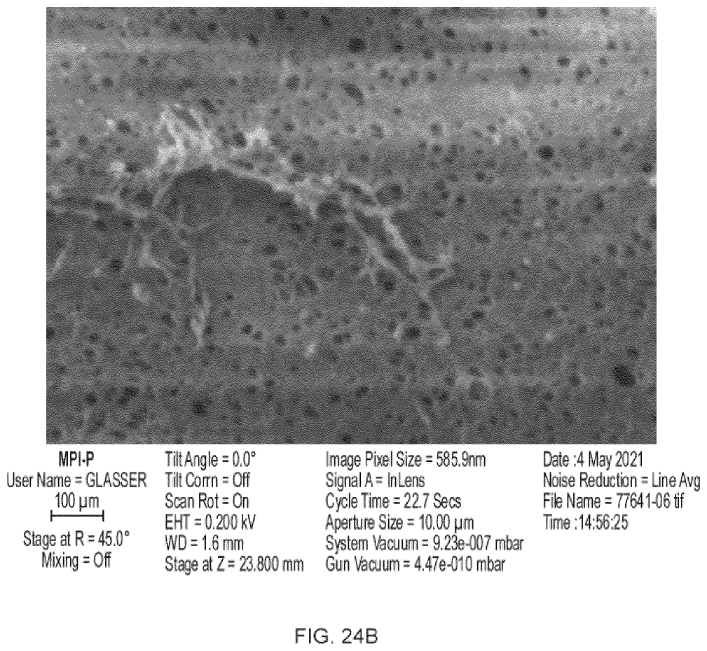

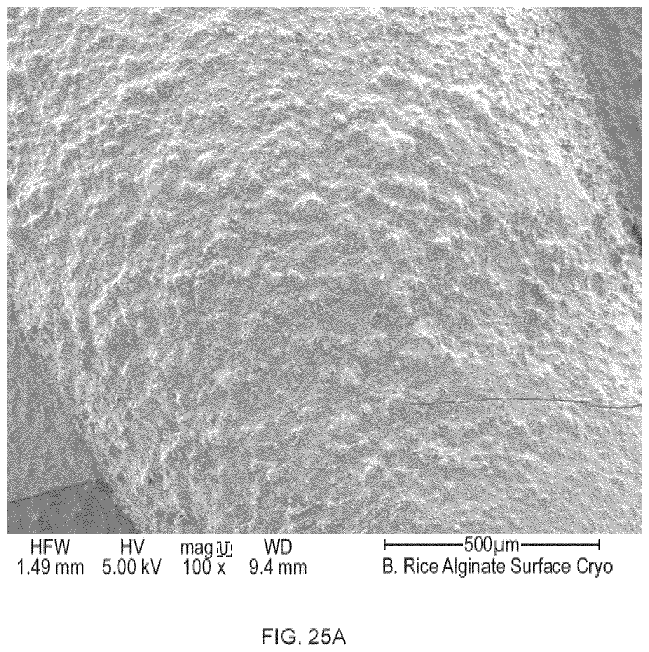

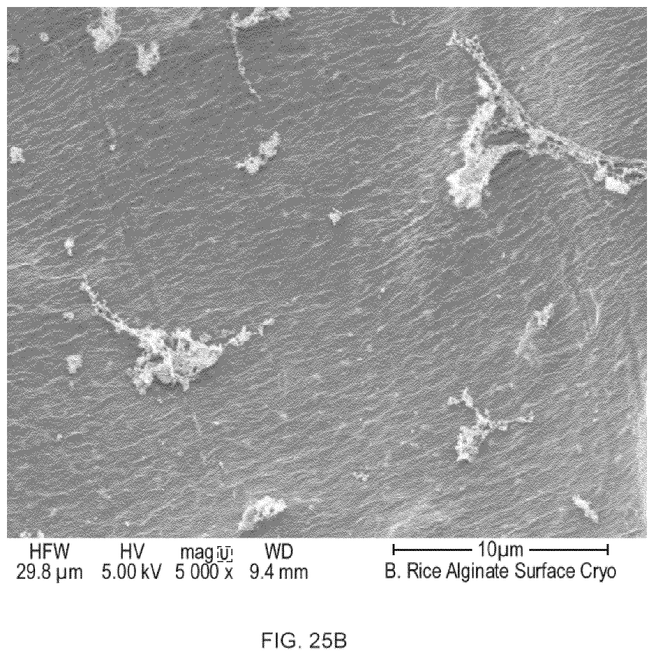

FIGS. 12-25 show SEMs show the porous nature of the edible hollow fibers of this example. It was unexpected that each of these mixes would have significantly different surface structures, skinning effects, surface porosity, as well as internal porosity. The plant-base protein isolates tended to have macro voids throughout the fiber surface, which are expected to be formed from the insoluble particles dislodging from the surface. These data also shows that, surprisingly and unexpectedly, the surface structure and pore size can be controlled by changing the source of protein or combining (I.e., one or more) proteins.

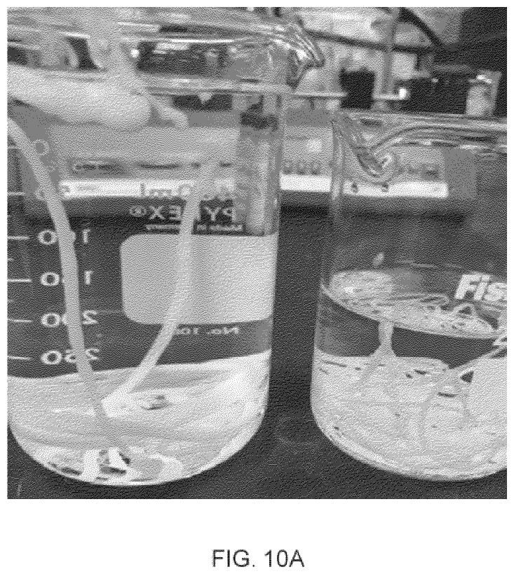

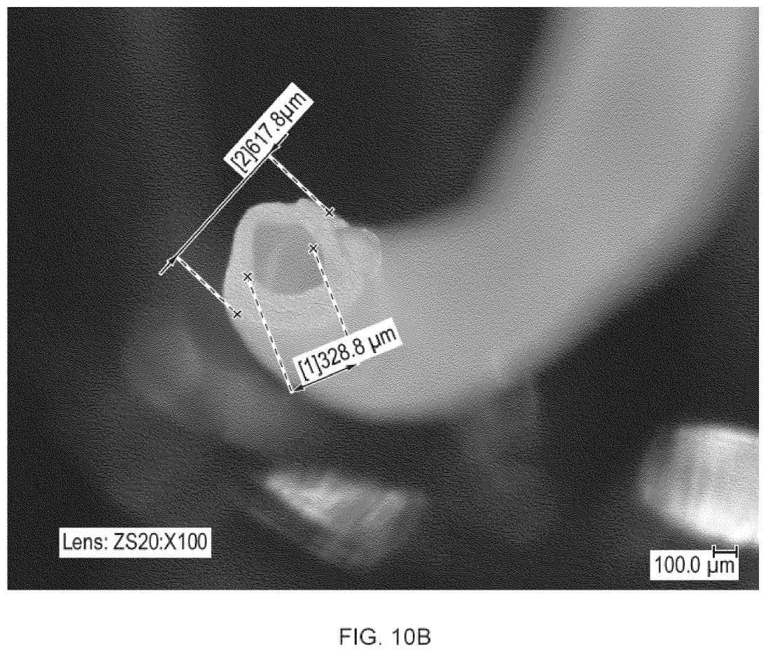

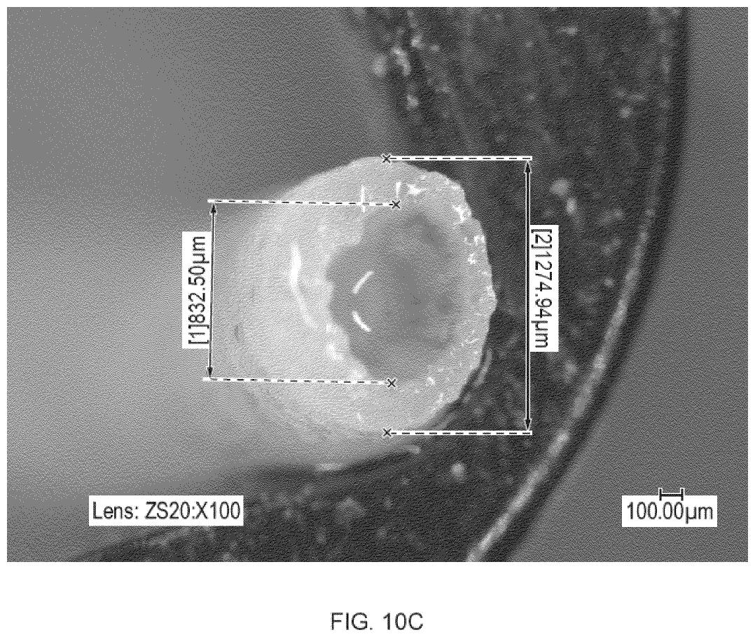

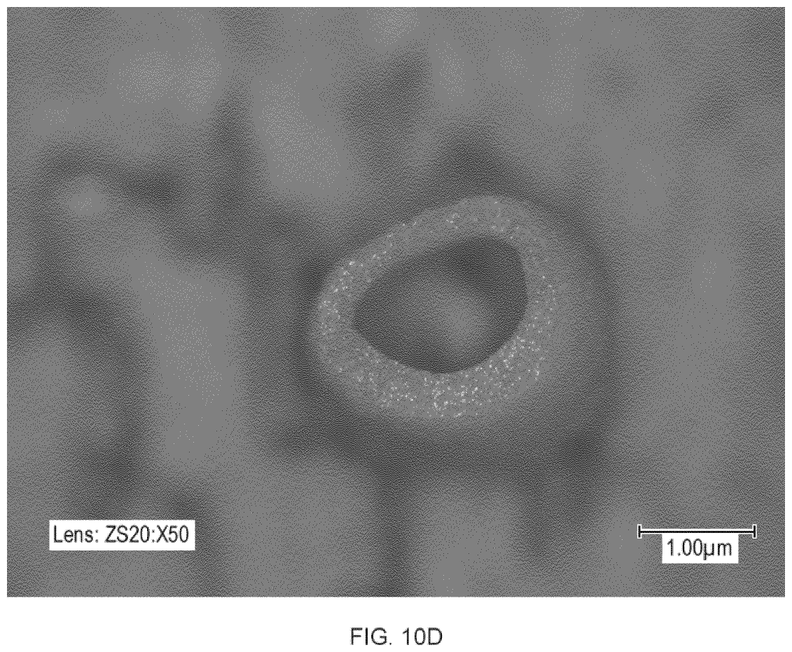

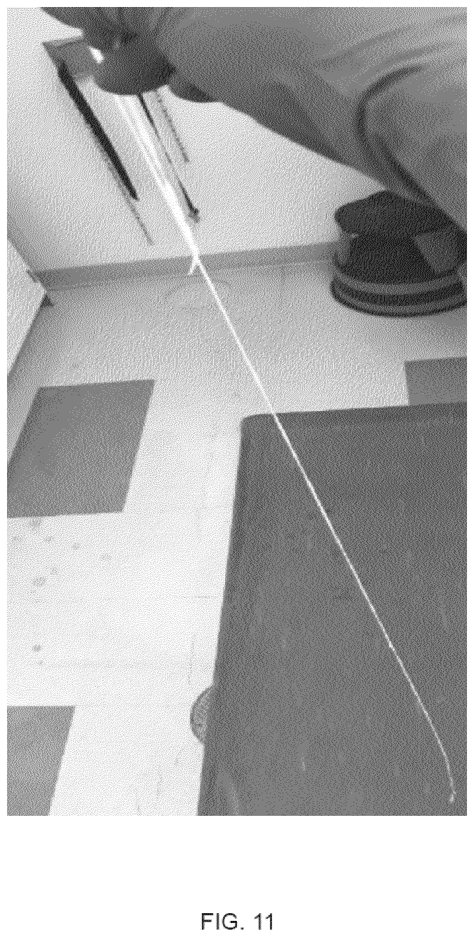

FIG. 10 shows fiber outer and inner diameter. The fiber outer and inner diameter was controlled via the dope flow rate through the coaxial needle. The dimensions of the fiber were tuned through nozzle diameters, flow rate, distance from the bath and, theoretically, the draw/tension on the fiber. FIG. 11 shows that the fibers made can easily support their wet weight as they will need to into a bioreactor. The example shown in FIG. 11 is of a 2 meter fiber, however much longer fibers can be made and can and support their own weight.

### Example 1C

Cell Growth on the Hollow Fibers of the Present Invention.

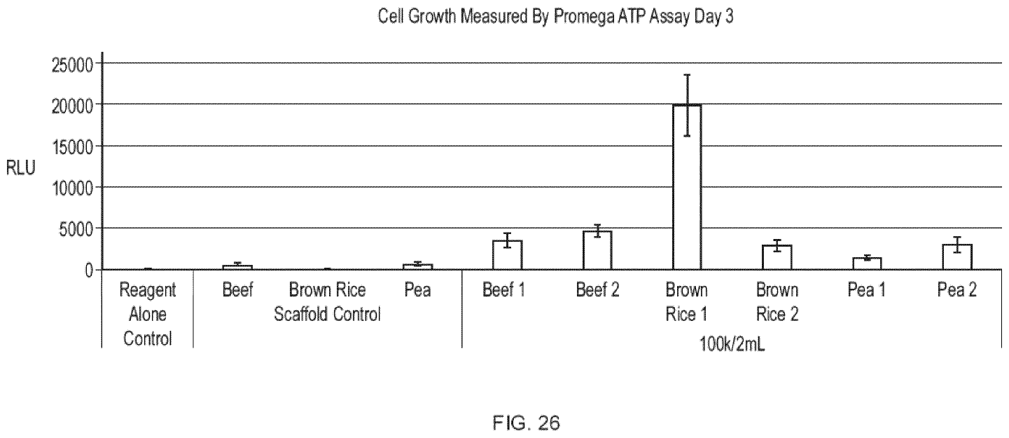

The hollow fibers comprising beef protein:alginate, brown rice protein:alginate or pea protein:alginate, made as detailed in Example 1B, were washed with PBS and incubated with DMEM/F12, lx glutamine and 10% fetal bovine serum (FBS) overnight in 15 ml conical tubes. C2C12 cells (murine muscle cell line, ATCC, Manassas, VA) were seeded at 100,000 cells per 2 ml. Cells were grown for 6 days and assayed at two time points, 3 and 6 days. Cell growth was assayed by ATP generation (CellTiter-Glo® 2.0 Cell Viability Assay, catalog no. G9421, Promega, Madison, WI). Readout was in raw luminescent units (RLU) and results were relative and linear. See, FIG. 26 where day 3 data is shown. Each hollow fiber formulation (beef:alginate, rice:alginate, pea:alginate) was produced on two different production runs and are denoted as 1 or 2 in the data. Each condition was run in triplicate. Bars indicate standard deviations. The brown rice:alginate mixture appears to support robust cell growth. The beef protein:alginate and pea protein:alginate mixtures also are supportive of cell growth.

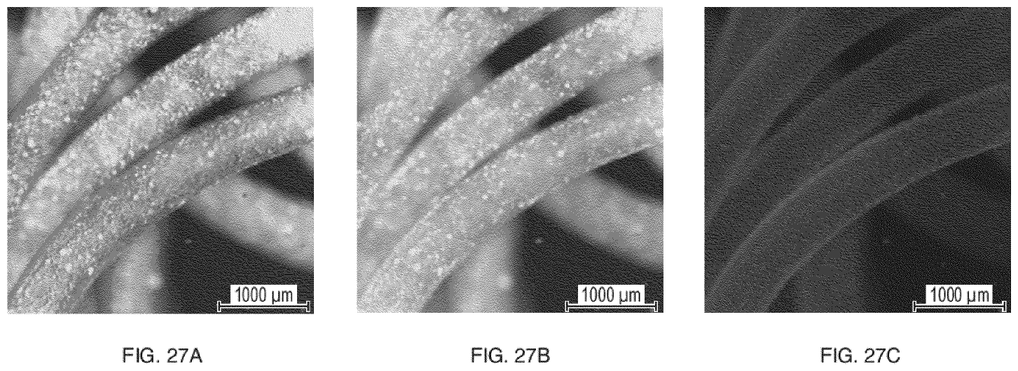

Cell growth and cell attachment on the brown rice protein; Aaginate hollow fibers made according to the protocols of the present invention are representative of the other fibers and production runs. FIG. 27 shows cell attachment and growth on hollow fibers made with brown rice; alginate 1 hollow fiber run on day 3 of the cell culture. FIG. 27A shows both staining of live cells and dead cells using the LIVE/DEAD™ Cell Imaging Kit (ThermoFisher, Waltham, MA) with live cells detected with CellTrace™ Calcein Green, AM and dead cells with BOBO™3 Iodide. FIG. 27B shows live cell staining with Calcein Green, AM and FIG. 27C shows dead cell staining with BOBO™3 of the same field of view. As is evident from this combined image, live cells make up the significant majority of the cell mass. This example shows that the hollow fibers of the present invention support cell attachment and growth.

### Example 2

Prophetic Exemplification of Design of the Cartridge of the Present Invention.

The cartridge is engineered to have specific fluid paths. The first fluid path has an inlet and outlet that allows the media to pass exclusively through the fibers. This fluid path is connected to a specific media reservoir. The second fluid path is designed for one-way flow around the fibers. The two inlets are on one endcap and the two outlets are on the other endcap. To create the homogenous flow, a core is centered in the cartridge where there's roughly the same number of fibers between the core wall and the shell of the cartridge. The length of the cartridge is designed based on the hollow fibers inner diameter and wall thickness, to optimize the homogeneity of the cell growth throughout the cartridge.

### Example 3

Prophetic Exemplification of the Assembly of the Hollow Fibers of the Present Invention into a Bioreactor of the Present Invention.

The device is considered a bioreactor after it is implemented into the entire cell growing system. The hollow fiber bioreactor is plumbed into the system with two separate fluid paths, as described in the previous example. The first described fluid path travels homogeneously around the fibers in the reactor. This fluid path is responsible for the seeding of the cells on the surface of the edible hollow fibers. After the seeding process is completed, this fluid path has a laminar flow of media through the cartridge and around the hollow fibers. This fluid path is adjustable to prevent the removal of cells from the fiber surface. The second fluid path is through the lumen of the hollow fibers. By balancing the fiber aspect ratio within the cartridge, fiber count, and flow rate, the cell density gradient is minimized, thereby allowing the formation of an essentially uniform cell mass.

### Example 4

Prophetic exemplification of a reactor utilizing an embodiment of the hollow fibers of the present invention. In this example a reactor comprising hollow fibers comprising alginate or similar material, having an outer diameter of 1.1 mm-1.2 mm and a wall thinness of 0.1 mm are assembled into a hollow fiber apparatus having approximately 83 fibers per cm³. The reactor is seeded with cells derived from iPSC and programed to differentiate into cells with myocyte morphological characteristics. The cells are seeded at a density of 10⁷/cm³. The cells seeded can be seeded while still in the expansion media or already in the media that promotes differentiation. After the cells are seeded, the media will be that of which a myocyte-like cell prefers. The reactor is sterilely connected to a media supply and operated for 14-21 days based on the seeding density and cell type, until cellular confluency is obtained, defined here as less than 10% free space between the fibers, and cells developed myocyte-like characteristics including forming myotubule-like structures adhered to the hollow fibers. At this point, the cell/hollow fiber mixture is removed from the reactor and for further processing, if any, and ultimately consumption.

### Example 5

Prophetic exemplification of a second reactor utilizing an embodiment of the hollow fibers of the present invention. In this example a reactor comprising hollow fibers comprising alginate or similar material, having an outer diameter of 1.1 mm-1.2 mm and a wall thinness of 0.1 mm are assembled into a hollow fiber apparatus having approximately 83 fibers per cm³. The reactor is seeded with cells derived from iPSC and programed to differentiate into cells with myocyte morphological characteristics or programmed to differentiate into cells with adipocyte characteristics at a ratio of about 90:10 cells programed to differentiate into cells with myocyte morphological characteristics: cells programmed to differentiate into cells with adipocyte characteristics. The cells are seeded at a total density of 10⁷/cm³. The reactor is sterilely connected to a media supply and operated for 14-21 days, until cellular confluency is obtained, defined here as less than 10% free space between the fibers, and cells developed myocyte-like characteristics including forming myotubule-like structures adhered to the hollow fibers. At this point, the cell/hollow fiber mixture is removed from the reactor and for further processing, if any, and ultimately consumption.

### Example 6

Prophetic exemplification of adding fats, flavors, etc., to the lumen of the hollow fibers after confluency of cell culture is reached. After the desired confluency is reached in the cultured structured clean meat product of the present invention, residual media is flushed out and the product infused with, for example, a saline solution. The concentration of the saline solution is determined by, for example, the desired salinity of the end product. The structured clean meat product of the invention is infused a higher concentration of salt(s) if a salted meat product is desired or a lower concentration of salt approaching zero if an unsalted meat product is desired. Additionally, fats, flavors, preservatives, etc., are infused into the void spaces and hollow fiber lumens of the cultured structured clean meat product of the present invention as desired and as determined by the final product.

### Example 7

Prophetic exemplification showing processing of harvested cells/hollow fibers. Once the cultured meat product (i.e., the “structured clean meat”) is produced by the culturing methods of the present invention and any desired flavors, fats or other additives are infused into the lumen of the hollow fibers and/or remaining void space, the hollow fiber cartridge of the present invention is removed from the housing. The hollow fiber cartridge then undergoes further processing. At least, the first and second end pieces are removed from the hollow fiber cartridge leaving the cultured structured clean meat product intact on the hollow fibers of the present invention. The structured clean meat product may then be sliced, textured, flavored or further flavored, etc., and packaged either for wholesale or retail sale, as desired.

We claim:

1) Edible hollow fibers, comprising one or more materials selected from the group consisting of hydrocolloids and proteins, having an outer diameter of about 0.2 mm to about 2.0 mm, a porosity of 0% to about 75% and a wall thickness of about 0.05 mm to about 0.4 mm.

2) The hollow fibers of claim 1, wherein said wall thickness is about 0.08 mm to 0.2 mm.

3) The hollow fibers of claim 1, wherein said porosity is about 40% to about 60%.

4) The hollow fibers of claim 1, wherein said hollow fibers comprise one or more of alginate, cellulose, chitosan, collagen, zein, agar, inulin, gluten, pectin, legume protein, methyl cellulose, pectin, gelatin, tapioca, xanthan gum, guar gum, tara gum, bean gum, plant protein, starch, plant isolates, lipids and phospholipids.

5) The hollow fibers of claim 4, wherein said proteins comprise one of more of corn protein, potato protein, wheat protein, sorghum protein, animal protein, animal protein isolate, beef protein isolate, casein protein and whey protein.

6) The hollow fibers of claim 4, wherein said plant isolates comprise one of more of soy, zein, casein, and wheat protein.

7) The hollow fibers of claim 4, wherein said lipids comprise one or more of free fatty acids, triglycerides, natural waxes and phospholipids.

8) The hollow fibers of claim 1, wherein said hollow fibers comprise one or more legume proteins and one or more hydrocolloids.

9) The hollow fibers of claim 1, each having a first end and a second end wherein said first end and said second end are positionally opposed to each other and, wherein a quantity of said hollow fibers are arranged in essentially in parallel and positioned such that the first ends of said hollow fibers are secured in a first holding device and the second ends of said hollow fibers are secured in a second holding device, the first and second holding devices being oriented essentially perpendicular to the longitudinal orientation of the hollow fibers and being orientated essentially parallel to each other, wherein at least one holding device allows for the flow of fluids to the interior of the hollow fibers, thereby creating a hollow fiber cartridge.

10) The hollow fiber cartridge of claim 9, wherein said hollow fibers are at a density of about 40-about 120 per cm².

11) The hollow fiber cartridge of claim 9, wherein said hollow fibers are at a density of about 60-about 100 per cm².

12) The hollow fiber cartridge of claim 9, wherein said hollow fibers are at a density of about 70-about 90 per cm².

13) The hollow fiber cartridge of claim 9 having a void space between the hollow fibers and, wherein the void space between the hollow fibers is about 25%-about 75% of the total volume of the hollow fiber cartridge.

14) The hollow fiber cartridge of claim 9, wherein the void space between the hollow fibers is about 40%-about 60% of the total volume of the hollow fiber cartridge.

15) The hollow fiber cartridge of claim 9, wherein said hollow fiber cartridge is designed to be removably inserted into a housing.

16) The hollow fiber cartridge of claim 15, wherein said housing is part of a bioreactor or bioreactor system.

17) A hollow fiber cell culture reactor, comprising;
a) the hollow fiber cartridge of claim 9;
b) a housing sized to hold said hollow fiber cartridge;
c) a medium source fluidly connected to one or more inlets in said housing;
d) one or more medium outlets in said housing; and,
e) one or more pumps to supply the medium to and/or remove waste medium from said hollow fiber cartridge through said medium inlet(s) and/or outlet(s).

18) The hollow fiber cell culture reactor of claim 17, wherein said inlets are fluidly connected to the interior of said hollow fibers.

19) The hollow fiber cell culture reactor of claim 17, further comprising an automated controller.

20) The hollow fiber cell culture reactor of claim 17, wherein the hollow fiber cartridge comprises hollow fibers at a density of about 20/cm² to about 100/cm².

21) The hollow fiber cell culture reactor of claim 17, wherein the hollow fiber cartridge comprises hollow fibers at a density of about 30/cm² to about 60/cm².

22) A process for producing a meat product, comprising; seeding the void space between the hollow fibers in said hollow fiber reactor of claim 17 with one or more of myocytes, myocyte-like cells or engineered cells expressing one or more myocyte-like characteristics at a density of about 10⁵ cells/ml to about 10⁸ cells/ml and culturing the cells until achieving about 80%-about 99% confluency.

23) The process of claim 22, additionally comprising removing said hollow fiber cartridge from the hollow fiber cell culture reactor after said cells have achieved about 80%-about 99% confluency.

24) The process of claim 23, additionally comprising removing said first holding device and said second holding device from the first ends and second ends, respectively, of said hollow fibers.

25) The process of claim 22, wherein said cells are cultured until achieving about 85%-about 99% confluency.

26) The process of claim 22, wherein said cells are cultured until achieving about 90%-about 99% confluency.

27) The process of claim 22, additionally comprising seeding the hollow fiber reactor with one or more of adipocytes, adipocyte-like cells or engineered cells expressing one or more adipocyte-like characteristics and/or fibroblasts, fibroblast-like cells or engineered cells expressing one or more fibroblast-like characteristics.

28) The process of claim 22, wherein said process additionally comprises supplying media to the cells through one or both of the first end and second end of the hollow fibers into the interior of the hollow fibers, through the wall of the hollow fibers into the void space between the hollow fibers where said cells are seeded and through one or more of said outlets in said housing.

29) The process of claim 22, further comprising, after the cells achieve confluency, infusing the interior of the hollow fibers and/or any remaining void space between the cells with one or more of fats, flavors, colors, salts and preservatives.

30) A structured clean meat product, comprising:
a) 50-90% cultured animal cells;
b) 10-30% edible hollow fibers and/or hollow fiber material;
c) 1-30% void space, said void space located between and/or interspersed with said cultured animal cells; and
d) 1-30% additives.

31) A structured clean meat product produced by the process of claim 22, comprising:
a) 50-90% cultured animal cells;
b) 10-30% edible hollow fibers and/or hollow fiber material;
c) 1-30% void space, said void space located between and/or interspersed with said cultured animal cells; and
d) 1-15% additives.

32) The structured meat product of claim 31, wherein said additives comprise one or more of flavors, texture enhancers, nutritional additives, preservatives and/or antioxidant, and fats and/or oils.

33) The structured clean meat product of claim 32, wherein said flavors are selected from one or more of essential oils, oleoresin (ESO), enzymes (ENZ), natural substances and extractives (NAT), non-nutritive sweetener (NNS), nutritive sweetener (NUTRS), spices, natural seasonings & flavorings (SP), and synthetic flavors (SY/FL), fumigant (FUM), artificial sweeteners and yeast extract.

34) The structured clean meat product of claim 32, wherein said texture enhancers are selected from one or more of pureed plant material, guar gum, cellulose, hemicellulose, lignin, beta glucans, soy, wheat, maize and rice isolates and beet fiber, pea fiber, bamboo fiber, plant derived fiber, plant derived gluten, carrageenan, xanthan gum, lecithin, pectin, agar, alginate, natural polysaccharides, grain husk, calcium citrate, calcium phosphates, calcium sulfate, magnesium sulfate and salts.

35) The structured clean meat product of claim 32, wherein said nutritional additives are selected from one or more of trace elements, bioactive compounds, endogenous antioxidants, A, B-complex, C, D, E vitamins, zinc, thiamin, riboflavin, selenium, iron, niacin, potassium, phosphorus, omega-3, omega-6, fatty acids, magnesium, protein, amino acids salt, creatine, taurine, carnitine, carnosine, ubiquinone, glutathione, choline, glutathione, lipoic acid, spermine, anserine, linoleic acid, pantothenic acid, cholesterol, Retinol, folic acid, dietary fiber and amino acids.

36) The structured clean meat product of claim 32, wherein said fats are selected from one or more of saturated, monounsaturated, polyunsaturated fats, corn oil, canola oil, sunflower oil, safflower oil, olive oil, peanut oil, soy bean, flax seed oil, sesame oil, canola oil, avocado oil, seed oils, nut oils, safflower and sunflower oils, palm oil, coconut oil, omega-3, fish oil, lard, butter, processed animal fat, adipose tissue, cellular agriculture derived fat essential oil and oleoresin.

37) The structured clean meat product of claim 31, wherein said hollow fibers comprise one or more legume proteins and one or more hydrocolloids.

38) The structured clean meat product of claim 31, wherein said void space is void of cells and/or cellular material.

39) The structured clean meat product of claim 32, wherein said preservatives and/or antioxidants are selected from one or more of: sodium salt, chloride salt, iodine salt. Nitrates, nitrosamines, butylated hydroxyanisole (BHA), butylated hydroxytoluene (BHT). sodium benzoate, potassium benzoate and benzene ascorbic acid, citric acid, potassium, monosodium glutamate (MSG), sulphur dioxide, sulphites, antibiotics.

40) The structured clean meat product of claim 31, wherein said void space is at least partly filled with material other than cells or cellular material.
